(19) United States
(12) Patent Application Publication
(10) Pub. No.: US 2022/0119829 A1
(43) Pub. Date: Apr. 21, 2022

(54) TRANSGENIC PLANTS AND METHOD OF FACILITATING TRANSFORMATION THEREOF

(71) Applicant: Purdue Research Foundation, West Lafayetter, IN (US)

(72) Inventors: Stanton B Gelvin, West Lafayette, IN (US); Lan-Ying Lee, West Lafayette, IN (US); Rachelle Amanda Lapham, West Lafayette, IN (US)

(73) Assignee: Purdue Research Foundation, West Lafayetter, IN (US)

(21) Appl. No.: 17/498,111

(22) Filed: Oct. 11, 2021

Related U.S. Application Data
(60) Provisional application No. 63/092,748, filed on Oct. 16, 2020.

(51) Int. Cl. C12N 15/82

(52) U.S. Cl. CPC C12N 15/8216

(57) ABSTRACT

The present disclosure provides transgenic plants and/or plant cells comprising overexpressed VirE2 gene or VirE2 protein in plant cytoplasm that upregulates or downregulates certain plant gene and/or proteins to facilitate transformation. The present disclosure further provides transgenic plants and/or plant cells comprising overexpressed plant gene or protein that upregulated by VirE2 gene or VirE2 protein for facilitating transformation. The transgenic plants and/or plant cells comprising downexpressed or knockout plant gene or protein that downregulated by VirE2 gene or VirE2 protein for facilitating transformation are also provided. Methods of making and using the transgenic plants and/or plants cells are also provided.

### CROSS-REFERENCE TO RELATED APPLICATIONS

This application claims the benefit of U.S. Provisional Application No. 63/092,748, filed on Oct. 16, 2020, which is incorporated herein by reference in its entirety.

### GOVERNMENT SUPPORT CLAUSE

This invention was made with government support under Award No. 1725122 awarded by the National Science Foundation. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 1, 2021, is named 941602-004U1_SL.txt and is 19,260 bytes in size.

### FIELD OF INVENTION

The present disclosure relates generally to transgenic plants and/or plant cells, and methods of facilitating transformation of plants and plant cells.

### BACKGROUND OF THE INVENTION

Agrobacterium tumefaciens, the causative agent of crown gall disease, transfers virulence effector proteins to infected host plants to facilitate the transfer of T-(transfer) DNA into and trafficking through plant cells. Once in the nucleus, T-DNA uses the host's machinery to express transgenes and may integrate into the host genome. Scientists have used this process to insert beneficial genes into plants by replacing native T-DNA genes with other genes of interest, making Agrobacterium-mediated transformation the preferred method for plant genetic engineering (Gelvin, 2003, 2012; Pitzchke and Hirt, 2010; Lacroix and Citovsky, 2013, 2019; Hiei et al., 2014; Nester, 2015; Van Eck, 2018).

VirE2 is one of the Agrobacterium tumefaciens effector proteins that is important for plant transformation (Gelvin, 2003, 2012). A. tumefaciens mutant strains lacking a functional virE2 gene are severely attenuated in virulence (Stachel and Nester, 1986), and integrated T-DNAs delivered from such strains often exhibit large deletions (Rossi et al., 1996). VirE2 can coat single-stranded DNA molecules in vitro (Gietl et al., 1987; Christie et al., 1988; Citovsky et al., 1988, 1989; Das, 1988; Sen et al., 1989) and has been proposed to coat single-stranded T-DNA molecules (T-strands) and protect them from nucleases as they traffic through the plant cell (Gietl et al., 1987; Citovsky et al., 1988; Tinland et al., 1994; Yusibov et al., 1994). Expression of VirE2 in the plant can complement a virE2 mutant Agrobacterium strain to full virulence (Citovsky et al., 1992; Simone et al., 2001), suggesting that one of VirE2's functions in transformation occurs in the plant and involves the maintenance of T-DNA integrity (Citovsky et al., 1988; Gietl et al., 1987).

VirE2 has been proposed to assist with nuclear import of T-strands through its interaction with the transcription factor VIP1 (VirE2-interacting protein 1; Tzfira et al., 2001). This observation led to the model that T-DNA-bound VirE2 binds VIP1 and uses VIP1 nuclear localization to deliver T-DNA into the nucleus (the “Trojan Horse” model; Djamei et al., 2007). However, conflicting reports of VirE2 subcellular localization exist in the literature (Citovsky et al., 1992, 1994, 2004; Tzfira and Citovsky, 2001; Tzfira et al., 2001; Li et al., 2005; Bhattacharjee et al., 2008; Grange et al., 2008; Lee et al., 2008; Shi et al., 2014; Lapham et al., 2018). In contrast to the Trojan Horse model, our laboratory showed that VirE2 holds at least a portion of the VIP1 pool outside the nucleus (Shi et al., 2014), and that VIP1 and its homologs are not required for Agrobacterium-mediated transformation (Shi et al., 2014; Lapham et al., 2018).

In addition to its proposed structural role in T-strand binding, other possible functions of VirE2 in transformation have been studied. VirE2 interacts with numerous plant proteins (Lee et al., 2008, 2012) including the transcription factors VIP1 and VIP2 (Tzfira et al., 2001; Anand et al., 2007; Pitzscke et al., 2009).

### SUMMARY OF THE INVENTION

The present disclosure transgenic plants or plant cells comprising one or more overexpressed plant genes or proteins that are upregulated by VirE2 gene or VirE2 protein in the cytoplasm to facilitate transformation. The present disclosure also provides transgenic plants or plant cells comprising downexpressed and/or knockout plant genes or proteins that are downregulated by VirE2 gene or VirE2 protein in the cytoplasm to facilitate transformation.

In certain embodiments, the upregulated plant genes or proteins include but are not limited to a transcription factor, such as WRKY33, an arabinogalactan protein (AGP) gene or protein, such as AGP17 or AGP31, a heat shock protein (HSP) transcript or protein, such as HSP90, a histone or histone modifying enzyme or its variants, such as histone H2A2 (HTA2) or its variants, histone H4 (HIS4 or HFO4), histone deacetylase HD2C (HDT3) or HDA3 (HDT1), and a cyclophilin protein, such as ROC2 or ROC3.

In certain embodiments, the downexpressed or knockout plant genes or proteins include but are not limited to alcohol dehydrogenase, such as ADH1, and a protein phosphatase 2C 25 (PP2C25).

The present disclosure further provides transgenic plants or plant cells comprising an overexpressed VirE2 gene or VirE2 protein in the plant cytoplasm that alters expression of a plant gene or protein to facilitate transformation in response to VirE2 gene or VirE2 protein induction. In certain embodiments, the present disclosure provides that the interactions of VirE2 with numerous plant proteins, including the transcription factors VIP1 and VIP2, leads to changes in plant gene expression and facilitates transformation which requires a cytoplasmic subcellular site of localization of VirE2. Plants expressing cytoplasmic localized VirE2-Venus or nuclear localized VirE2-Venus-NLS were generated under the control of a β-estradiol inducible promoter. Following induction, these plants were assayed for transformation using a virE2 mutant Agrobacterium strain. Only cytoplasmic localized VirE2 supports transformation, indicating that VirE2's major function in transformation occurs in the cytoplasm.

In certain embodiments, the present disclosure provides RNA-seq and proteomic analyses that were also performed on transgenic Arabidopsis thaliana roots before and after VirE2 expression. Genes previously shown to be important for transformation were differentially expressed in the presence of VirE2, and proteins known to be important for transformation were more prevalent after VirE2 induction, facilitating transformation. Knockout mutant lines of some of the differentially expressed genes exhibited altered transformation phenotypes. Transgenic plants overexpressing cDNAs encoding some of the proteins shown to be more prevalent in the presence of VirE2 had enhanced transformation susceptibility.

In certain embodiments, the present disclosure provides that overexpressing a VirE2 gene and/or a VirE2 protein in the plant cytoplasm facilitates transformation of such plant by upregulating certain plant genes and/or proteins. These upregulated plant genes and/or proteins include but are not limited to transcription factors, such as WRKY33, arabinogalactan proteins (AGPs), such as AGP17 or AGP31, heat shock proteins (HSP), such as HSP90, histones or histone modifying enzymes, such as histone H2A2 (HTA2) or its variants, histone H4 (HIS4 or HFO4), histone deacetylase HD2C (HDT3) or HDA3 (HDT1), and cyclophilin proteins, such as ROC2 or ROC3.

The present disclosure further provides that overexpressing certain plant genes and/or proteins induced by VirE2 gene facilitates transformation of a plant. The identified plant genes and/or proteins induced by VirE2 gene include but are not limited to transcription factors, such as WRKY33, arabinogalactan proteins (AGPs), such as AGP17 or AGP31, heat shock proteins (HSP), such as HSP90, histones or histone modifying enzymes, such as histone H2A2 (HTA2) or its variants, histone H4 (HIS4 or HFO4), histone deacetylase HD2C (HDT3) or HDA3 (HDT1), and cyclophilin proteins, such as ROC2 or ROC3.

In other embodiments, the present disclosure provides that overexpressing a VirE2 gene and/or a VirE2 protein in the plant cytoplasm facilitates transformation of such plant by downregulating certain plant genes and/or proteins. Therefore, the present disclosure further provides that downregulating the expression of certain plant genes and/or proteins facilitates transformation of a plant. These downregulated plant genes and/or proteins include but are not limited to defense or stress-response genes or proteins, such as alcohol dehydrogenase (ADH1) or a protein phosphatase 2C-25 (PP2C25).

In certain embodiments, the plant is Arabidopsis plant and/or plant cells comprising Agrobacterium VirE2 gene or VirE2 protein to faciliating Agrobacterium-mediated transformation (AMT).

Methods of facilitating transformation of a plant or plant cell comprising overexpressing a VirE2 gene or a VirE2 protein in the plant cytoplasm are provided herein. Furthermore, methods of facilitating transformation of a plant or plant cell comprising overexpressing one or more plant gene or protein that is upregulated by VirE2 gene or VirE2 protein, and/or down-expressed or knockout a plant gene or protein that is downregulated by VirE2 gene or VirE2 protein, are also provided.

### BRIEF DESCRIPTION OF THE DRAWINGS

FIGS. 1A-1D. Subcellular localization of VirE2-Venus (FIGS. 1A-1B) and VirE2-Venus-NLS (FIGS. 1C-1D) in A. thaliana roots. Transgenic A. thaliana plants expressing inducible VirE2-Venus or VirE2-Venus-NLS were treated with β-estradiol (FIG. 1A & FIG. 1C) or control solution (FIG. 1B & FIG. 1D). Cerulean-NLS under the control of a CaMV 2x35S promoter was used to mark the nuclei. Root cells were imaged by confocal microscopy 9 hrs after treatment and representative images are shown. Four images of each cell are presented (left to right: Merged DIC+YFP+Cerulean; Cerulean; Venus; merged Venus+Cerulean). Boxes indicate an enlargement of one portion of the merged Venus+Cerulean image. Bars indicate 100 μm.

FIGS. 2A-2B. Transformation susceptibility of Arabidopsis wild-type (Col-0) and β-estradiol inducible transgenic VirE2-Venus and VirE2-Venus-NLS plants. Agrobacterium-mediated transient transformation assays were conducted on roots of three transgenic lines of inducible VirE2-Venus, three transgenic lines of inducible VirE2-Venus-NLS, and wild-type Col-0 plants. Following treatment for 24 hr with β-estradiol or control solutions, root segments were inoculated with (FIG. 2A) 10⁸ cfu/mL of the virE2 mutant strain A. tumefaciens At1879 containing pBISN2 or (FIG. 2B) 10⁵ cfu/mL of the wild-type VirE2 strain EHA105::pBISN1 (At1529). Root segments were stained with X-gluc six days after infection. Bars represent an average of three biological replicates (each replicate containing >60 root segments)+SE. ANOVA test *P value <0.05, **P value <0.01, ns, not significant.

FIGS. 3A-3B. Gene Ontology (GO) Biological Process Categories of up- (FIG. 3A) and down-regulated (FIG. 3B) genes in the presence of VirE2. Displayed are categories of genes with 1.3-fold or greater change in expression, considering all time points.

FIG. 4. Gene Ontology (GO) Enrichment Analysis of VirE2 differentially expressed genes. GO biological processes of over-represented gene categories for VirE2 differentially expressed genes at all time points. Displayed are results only with a false discovery rate (FDR)<0.05.

FIGS. 5A-5E. Quantitative RT-PCR analysis of selected VirE2 differentially expressed genes in inducible VirE2-Venus (cytoplasmic) versus inducible VirE2-Venus-NLS (nuclear) plants. VirE2-Venus (left) and VirE2-Venus-NLS (right) results of (FIG. 5A) FRO2, (FIG. 5B) TMP, (FIG. 5C) HSP90, (FIG. 5D) LEA4-5, and (FIG. 5E) CBFP gene expression in induced relative to non-induced roots. Bars represent an average of three technical replicates ±SE for one representative biological replicate of one transgenic line. Relative expression is shown after 3 (LEA4-5 only) or 12 hours after induction in the presence of A. tumefaciens A136.

FIGS. 6A-6D. Gene Ontology (GO) Biological Process Categories of VirE2 differentially expressed proteins. Proteins are grouped according to Gene Ontology (GO) process terms. Up-regulated proteins after 3 (FIG. 6A) or 12 (FIG. 6B) hours of VirE2 induction are shown along with down-regulated proteins after 3 (FIG. 6C) or 12 (FIG. 6D) hours of VirE2 induction. Only proteins which showed at least a 20% change in abundance for all three biological replicates determined by two different computational methods are shown. Total protein number is shown in the upper right corner of each graph and is highlighted in gray (up-regulated) or in black (down-regulated).

FIGS. 7A-7B. Subcellular localization of VirE2-Venus (FIG. 7A) and VirE2-Venus-NLS (FIG. 7B) in tobacco BY-2 protoplasts. A total of 10 μg of DNA encoding VirE2-Venus or VirE2-Venus-NLS was cotransfected with 10 μg of DNA encoding a nuclear marker mRFP-NLS into tobacco BY-2 protoplasts. Cells were imaged by confocal microscopy 16 hrs after transfection and representative images are shown. Four images of each cell are presented (left to right: DIC; mRFP; YFP; merged YFP+mRFP). We examined at least ten cells per experiment and performed each experiment three times. The same localization patterns each time was observed. Bars indicate 10 μm.

FIGS. 8A-8B. Expression kinetics of VirE2 measured by RT and RT-qPCR. FIG. 8A. A 250 bp PCR product was amplified from the 3′ end of VirE2 transcripts and visualized by ethidium bromide staining after electrophoresis through a 1.5% agarose gel. Samples were harvested 0, 1, 3, 6, 12, and 24 h post-induction with β-estradiol. As a control for RNA integrity, a 211 bp PCR product was amplified from ACTIN2 (ACT2) transcripts. M, size marker; FIG. 8B. Quantitative RT-PCR of VirE2 gene expression in induced relative to non-induced roots in the presence of A. tumefaciens A136. Results show the average of three technical replicates ±SE. Relative expression is shown after 3 and 12 hr. ANOVA test: *P-value <0.05, **P-value <0.01, ***P-value <0.001.

FIGS. 9A-9H. Quantitative RT-PCR of selected VirE2 Differentially Expressed Genes. RNA-seq (left) and quantitative RT-PCR (right) results of (FIG. 9A) ADH1 (FIG. 9B) PRKP (FIG. 9C) TAS4, (FIG. 9D) PR, (FIG. 9E) LSU1, (FIG. 9F) LRRPK, (FIG. 9G) AGP21, and (FIG. 9H) NTR2.6 gene expression in induced relative to non-induced roots. Results represent an average of three replicates ±SE for inducible VirE2 Line #10. Relative expression is shown 3 and 12 hours after induction in the presence of A. tumefaciens A136. ANOVA test: *P-value <0.05, **P-value <0.01, ***P-value <0.001.

FIGS. 10A-10H. Transformation susceptibility of Arabidopsis wild-type (Col-0) and T-DNA insertion mutant plants of VirE2 up-regulated genes. Agrobacterium-mediated transient or stable transformation assays were conducted on Col-0, IncRNA (FIG. 10A), atpsk3, acs6 (FIG. 10B), tst18 (FIG. 10C), pry (FIG. 10D), agp14 (FIG. 10E), tasi4 (FIG. 10F), miR163, samp (FIG. 10G), and tasi3 (FIG. 10H) mutant plants. Root segments were inoculated with 107, 106, or 105 cfu/mL of A. tumefaciens At849 (transient) or A208 (stable). For the transient assay, the root segments were stained with Xgluc 6 days after infection. For stable transformation, tumors were scored 30 days after infection. Numbers represent an average of three biological replicates (each replicate containing >60 root segments)+SE. ANOVA test *P-value <0.05, **P-value <0.01, ns: not significant. The data are shown only if the transformation efficiency was ≥5%.

FIGS. 11A-11F. Transformation susceptibility of Arabidopsis wild-type (Col-0) and T-DNA insertion mutant plants of VirE2 down-regulated genes. Agrobacterium-mediated transient or stable transformation assays were conducted on Col-0, ex11 (FIG. 11A), mee39, rbc3b, abah3 (FIG. 11B), ntr2.6, cup (FIG. 11C), ntr2.1, oep6 (FIG. 11D), esm1, rld17 (FIG. 11E), pp2c25, and adh1 (FIG. 11F) mutant plants. Root segments were inoculated with 107 or 106 cfu/mL of A. tumefaciens At849 (transient) or A208 (stable). For the transient assay, the root segments were stained with X-gluc 6 days after infection. For stable transformation, tumors were scored 30 days after infection. Numbers represent an average of two or three biological replicates (each replicate containing >60 root segments)+SE. ANOVA test *P-value <0.05, **P-value <0.01, ***P-value <0.001, ns: not significant.

FIGS. 12A-12E. Transformation susceptibility of Arabidopsis overexpression plants of genes whose protein levels are increased in response to VirE2 relative to wildtype (Col-0). Agrobacterium-mediated transformation assays were conducted on Col-0, PERX34 (FIG. 12A), ROC2 (FIG. 12B), HDA3 (FIG. 12C), HD2C (FIG. 12D), and AGP31 (FIG. 12E) overexpression plants. Numbers of the x-axis represent independent transgenic over-expression lines (T2 generation). Root segments were inoculated with 107 cfu/ml or 106 cfu/mL of A. tumefaciens At849 (transient) and A208 (stable). For the transient assay, the root segments were stained with X-gluc 6 days after infection. For stable transformation, tumors were scored 30 days after infection. Bars represent an average of three biological replicates (each replicate containing >60 root segments)+SE. ANOVA test *P-value <0.05, **P-value <0.01, ***P-value <0.001.

### DETAILED DESCRIPTION OF THE INVENTION

The present disclosure provides transgenic plants and/or plant cells comprising VirE2 gene or VirE2 protein, and methods of potentiating transformation of plants and plant cells comprising VirE2 gene or VirE2 protein to induce certain plant genes and/or proteins to facilitate transformation of such plant. The present disclosure provides that VirE2 is localized in the plant cytoplasm and then alters expression of specific plant genes and proteins to facilitate transformation.

The present disclosure also provides transgenic plants and/or plant cells, and method of making and using thereof, comprising overexpressed one or more plant genes and/or proteins that are upregulated by VirE2 gene or VirE2 protein. The present disclosure further provides transgenic Arabidopsis plants and/or plant cells comprising down-expressed and/or knockout one or more plant genes and/or proteins that are downregulated by VirE2 gene or VirE2 protein.

In certain embodiments, the identified plant genes and/or proteins induced by VirE2 gene to facilitate transformation include but are not limited to transcription factors, such as WRKY33, arabinogalactan proteins (AGPs), such as AGP17 or AGP31, heat shock proteins (HSP), such as HSP90, histones or histone modifying enzymes, such as histone H2A2 (HTA2) or its variants, histone H4 (HIS4 or HFO4), histone deacetylase HD2C (HDT3) or HDA3 (HDT1), and cyclophilin proteins, such as ROC2 or ROC3.

In other embodiments, the identified plant genes and/or proteins that are downregulated by VirE2 gene or protein include but are not limited to defense or stress-response genes or proteins, such as alcohol dehydrogenase (ADH1) or a protein phosphatase 2C-25 (PP2C25).

In certain embodiments, the plant is Arabidopsis plant and/or plant cells comprising Agrobacterium VirE2 gene or VirE2 protein to facilitating Agrobacterium-mediated transformation (AMT).

The following description of the embodiments is merely exemplary in nature and is in no way intended to limit the present disclosure, its application, or uses. Although specific terms are employed herein, they are used in a generic and descriptive sense only and not for purposes of limitation.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing certain aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a short chain fatty acid,” “a carnitine derivative,” or “an adjuvant,” includes, but is not limited to, combinations of two or more such short chain fatty acids, carnitine derivatives, or adjuvants, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Transgenic Arabidopsis plants and/or plant cells comprising Agrobacterium VirE2 gene and methods of potentiating Agrobacterium-mediated transformation (AMT) of plants and plant cells comprising VirE2 gene are provided herein. The present disclosure provides that only cytoplasmic localized VirE2-Venus, but not nuclear localized VirE2-Venus-NLS, complements the loss of virulence of a virEZ Agrobacterium mutant, suggesting that the major role of VirE2 in transformation occurs in the cytoplasm.

The reported subcellular localization of VirE2 is controversial. When tagged on its N-terminus, VirE2 was reported to localize to the nucleus (Citovsky et al., 1992, 1994, 2004; Tzfira and Citovsky, 2001; Tzfira et al., 2001; Li et al., 2005). Other studies showed that both N- and C-terminally tagged VirE2 localized to the cytoplasm (Bhattacharjee et al., 2008; Grange et al., 2008; Lee et al., 2008; Shi et al., 2014; Lapham et al., 2018). However, only the C-terminally tagged fusion protein, when expressed in a plant, could complement a virE2 mutant strain and restore efficient transformation (Bhattacharjee et al., 2008).

More recently, Li et al. (2014) showed that an Agrobacterium strain expressing VirE2 with an internal small GFP fragment (GFP11) is virulent. Using this strain and a split-GFP approach, VirE2-GFP11 delivered from Agrobacterium could refold with GFP1-10 expressed in planta to restore GFP fluorescence. In the plant cell, VirE2-GFP complexes formed filamentous structures mainly in the cytoplasm and with a few that appeared within the nucleus. Roushan et al. (2018) used phiLOV2.1 to tag VirE2 internally and showed that, when transferred from Agrobacterium, the protein localized to the cytoplasm of Arabidopsis roots and N. tabacum leaves. Li et al. (2020) further demonstrated that only very small amounts of VirE2 could be detected in the nucleus in the presence of VirD2 and T-strands, solving the conundrum of conflicting results from different laboratories. The studies presented herein provide that, regardless of its site of synthesis, only when VirE2-Venus protein localizes to the cytoplasm can it complement a virE2 mutant Agrobacterium strain. An inducible nuclear-localized VirE2-Venus-NLS protein could not complement the virE2 mutant strain. These results confirm previous observations (Bhattacharjee et al., 2008) and indicate that VirE2 must localize to the cytoplasm to perform its functions in Agrobacterium-mediated transformation.

VirE2 interacts with several Arabidopsis importin α (Impα) isoforms in a yeast two hybrid system and in plant cells when overexpressed (Bhattacharjee et al., 2008, Lee et al., 2008). VirE2 interacts with many Impα isoforms in the cytoplasm, but only VirE2-Impα-4 interaction localizes to the nucleus of BY-2 protoplasts. Although VirE2 protein contains two putative bipartite NLS sequences (Citovsky et al., 1992, 1994), structural analyses indicated that the interactions between rice Impα1α and the VirE2 NLS sequences are weak (Chang et al., 2017). Ziemienowicz et al. (2001) observed that VirE2 bound to ssDNA was not imported into isolated tobacco nuclei, but they did observe the import of free VirE2 molecules into the nucleus. On the other hand, VirE2, in addition to the effector protein VirD2, was required for nuclear import of large ssDNA molecules in this in vitro system (Ziemienowicz et al., 2001). It is possible that a small amount of VirE2 localizes to the nucleus during transformation. However, based on the studies presented herein, exclusive nuclear localization of VirE2 does not support transformation.

The present disclosure provides studies for functions of VirE2 in transformation other than its proposed structural roles in protecting T-strands (Howard and Citovsky, 1990) and/or shaping T-strands to traverse the nuclear pores (Ziemienowicz et al., 2001; Li et al., 2020). VirE2 interacts with the Arabidopsis transcription factors VIP1 and VIP2 (Tzfira et al., 2001; Anand et al., 2007; Pitzschke et al., 2009) and various other plant proteins (Lee et al., 2008, 2012). Although VIP1 and its orthologs do not play a role in Agrobacterium-mediated transformation (Shi et al., 2014; Lapham et al., 2018), interactions with VIP2 or other proteins lead to changes in plant gene expression and facilitate transformation.

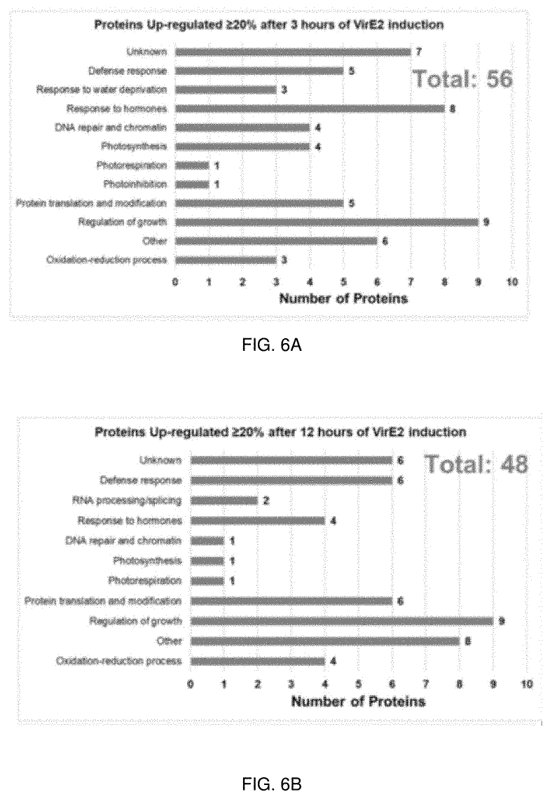

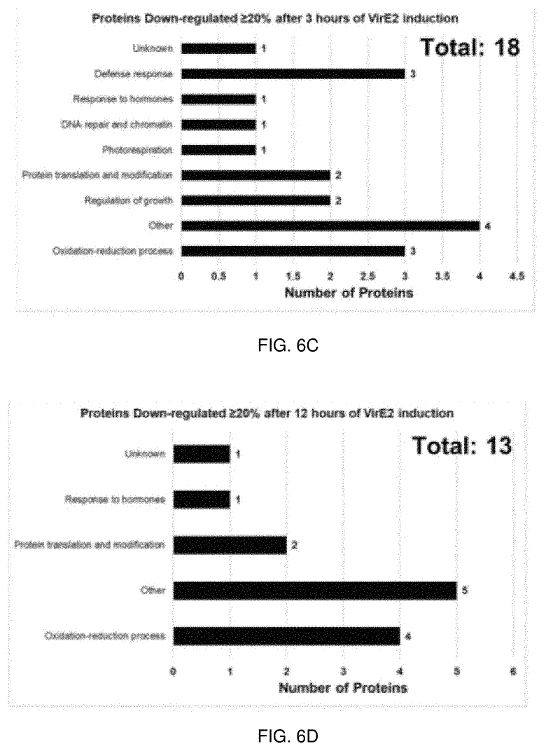

RNA-seq analysis of transgenic Arabidopsis thaliana roots expressing VirE2 revealed that most transcript abundance changes occurred 12 hours post-VirE2 induction. Conversely, proteomics analysis indicated that numerous proteins changed abundance 3 hours after VirE2 induction, but none of the transcripts for these proteins changed abundance at that early time. These results suggest that alterations in mRNA and protein abundance in response to VirE2 expression occur post-transcriptionally, most likely at the translational or post-translational level. This finding is consistent with cytoplasmic-rather than nuclear-localized VirE2. It is also supported by the data showing that proteins involved in translation also exhibited rapid changes in their steady-state levels in response to VirE2 induction (FIGS. 6A-6D).

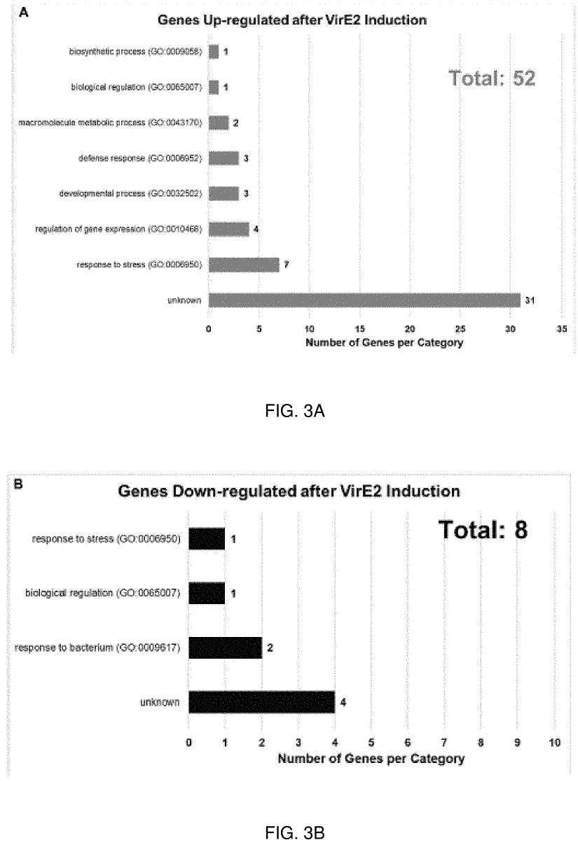

Genes involved in plant defense were differentially expressed in response to VirE2 induction (FIGS. 3A-3B). Duan et al. (2018) noted that expression of several defense genes was upregulated in A. thaliana constitutively expressing VirE2 24 hours after the plants were treated with the avirulent Agrobacterium strain A136. They also found that plants constitutively expressing VirE2 had reduced transformation efficiency compared to wild-type plants. This inhibition may be caused by enhanced defense responses in the VirE2-expressing plants. Upregulation of genes involved in innate immune responses was also observed 12 hours after VirE2 induction in the presence of the avirulent Agrobacterium strain A136 (FIGS. 3A-3B; Supplemental Data Sheet 1 and 2), but the genes identified in the studies of the present disclosure differed from those identified previously by Duan et al. (2018). Ditt et al. (2006) found that genes involved in response to biotic stimulus, abiotic stimulus, and stress were enriched for transcripts up-regulated 48 hours after infection of Arabidopsis cell cultures (ecotype Ler) by the tumorigenic Agrobacterium strain A348. Upregulation of these same gene categories was observed 12 hours after VirE2 induction in the presence of the avirulent Agrobacterium strain A136. Veena et al. (2003) observed an increase in defense response gene transcripts early (3-6 hours) after Agrobacterium infection of N. tabacum BY-2 suspension cells, but expression of these genes was suppressed at later infection times (30-36 hours) in the presence of Agrobacterium strains that could transfer virulence proteins. However, suppression of this delayed defense response did not occur when the plants were infected with the transfer-deficient Agrobacterium strain A136 (Veena et al., 2003).

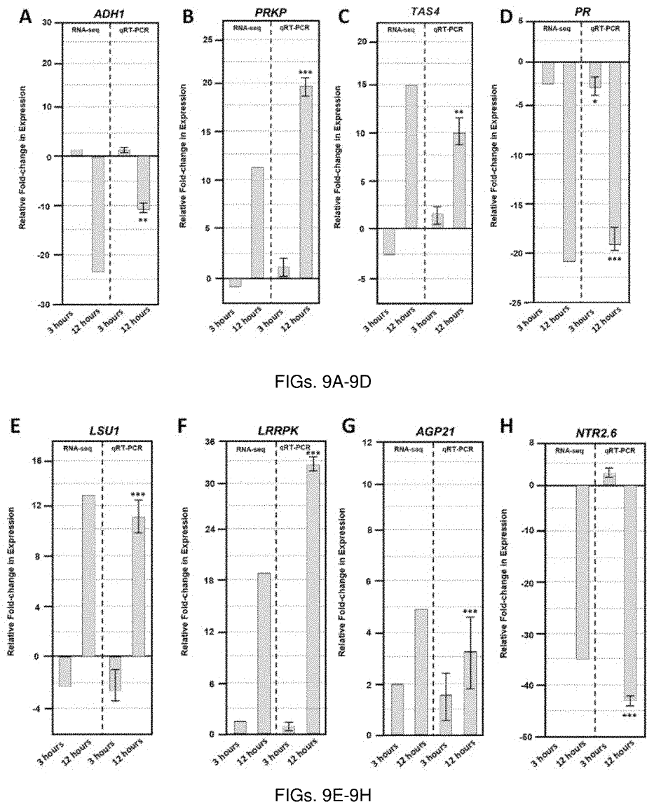

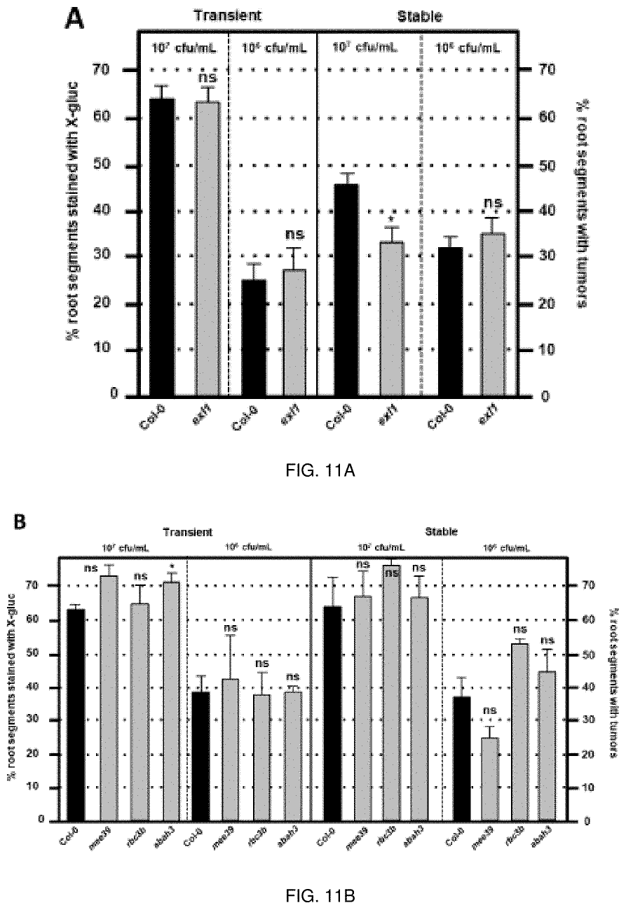

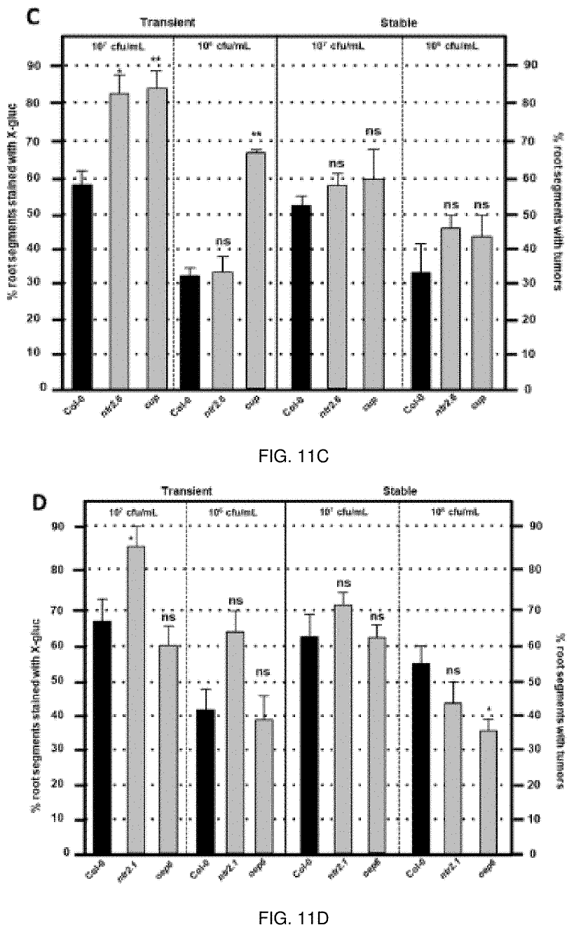

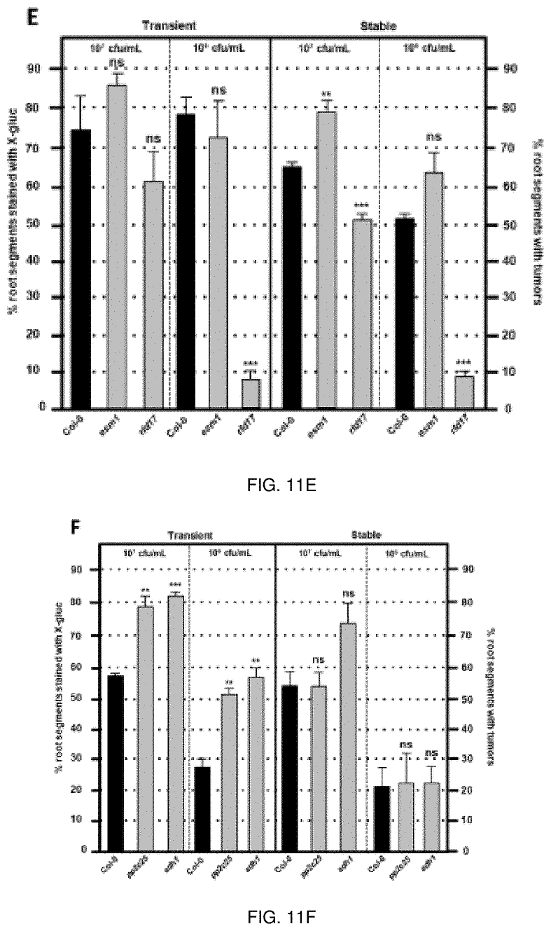

The stress-response associated alcohol dehydrogenase 1 (ADH1) gene was strongly downregulated in the presence of VirE2 (Table 2; FIG. 9A) and a knockout mutant line of this gene showed increased transformation (FIG. 11F). Veena et al. (2003) also found that a tobacco alcohol dehydrogenase gene was downregulated in the presence of a virulent Agrobacterium strain at later infection time points. In addition, the RNA-seq experiments revealed that the transcription factor WRKY33 was upregulated 12 hours after VirE2 induction. Zheng et al. (2006) showed that ectopic overexpression of WRKY33 resulted in increased susceptibility to the bacterial pathogen Pseudomonas syringae, and that WRKY33 could act as a negative regulator of bacterial defense responses.

Genes known to be important for transformation, including those encoding a protein phosphatase 2C (Tao et al., 2004), arabinogalactan proteins (Nam et al., 1999; Gaspar et al., 2004), and heat shock proteins (Park et al., 2014), showed changes in expression in response to VirE2. Protein phosphatase 2C 25 (PP2C25) was downregulated by VirE2 (Table 2) and its knockout mutant line exhibited increased transformation (FIG. 11F). A tomato protein phosphatase 2C (DIG3) was previously shown to act as a negative regulator of transformation by dephosphorylating a serine residue in VirD2 that is critical for VirD2 nuclear import (Tao et al., 2004). VirE2-mediated down-regulation of PP2C25 may therefore facilitate more efficient nuclear import of VirD2/T-strand complexes.

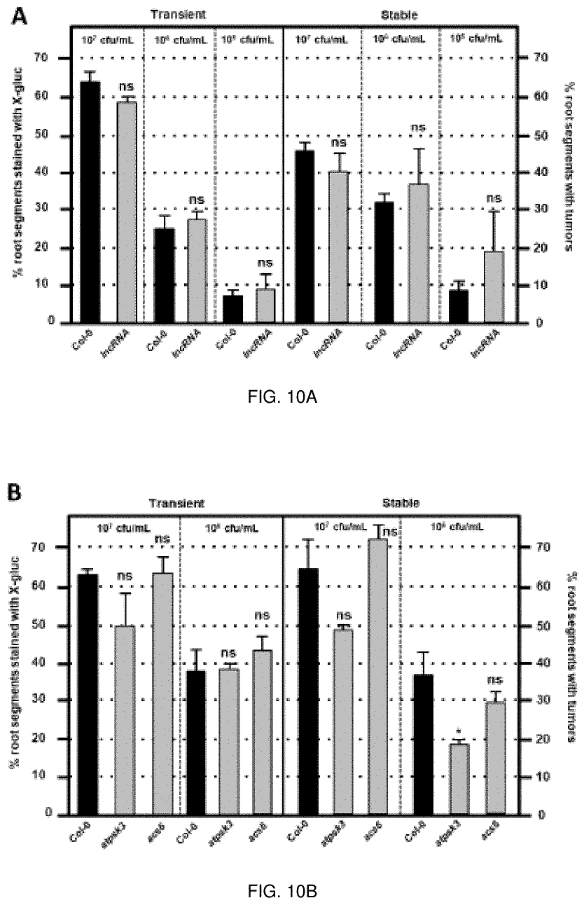

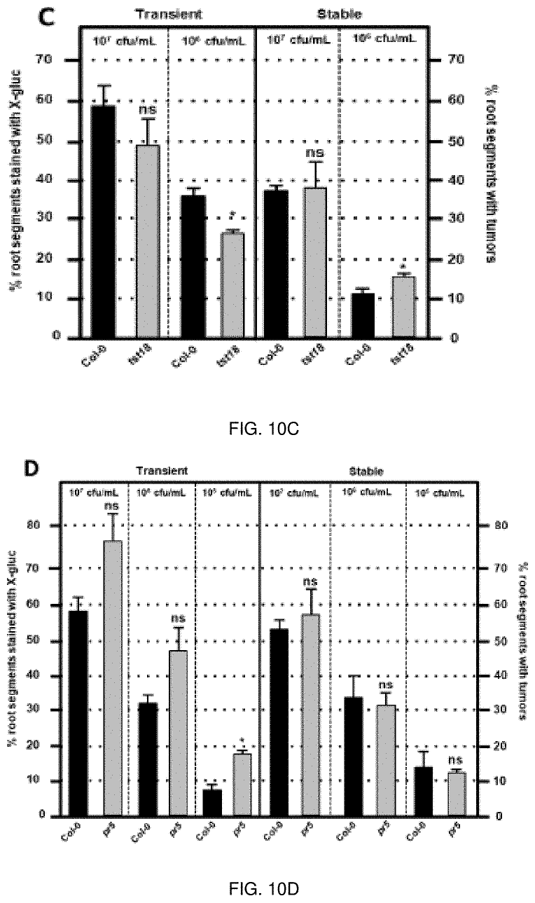

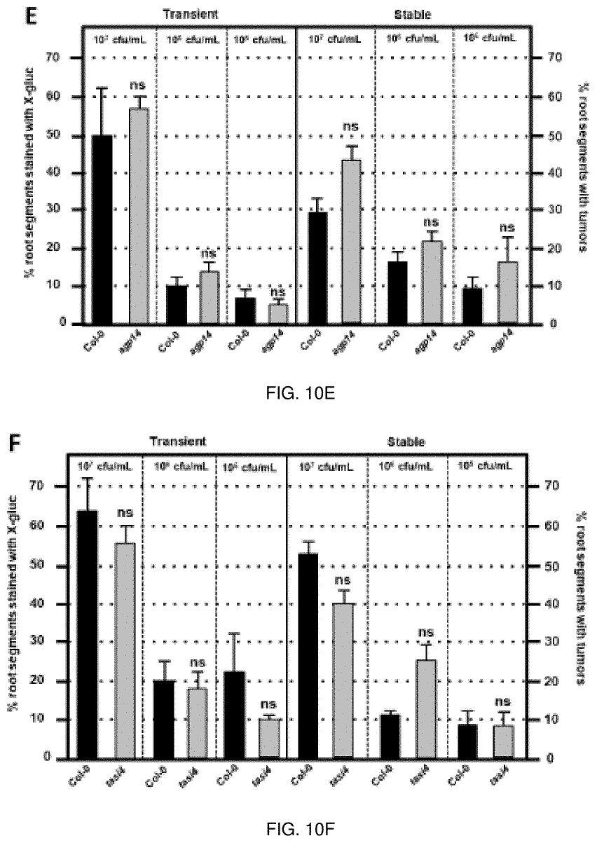

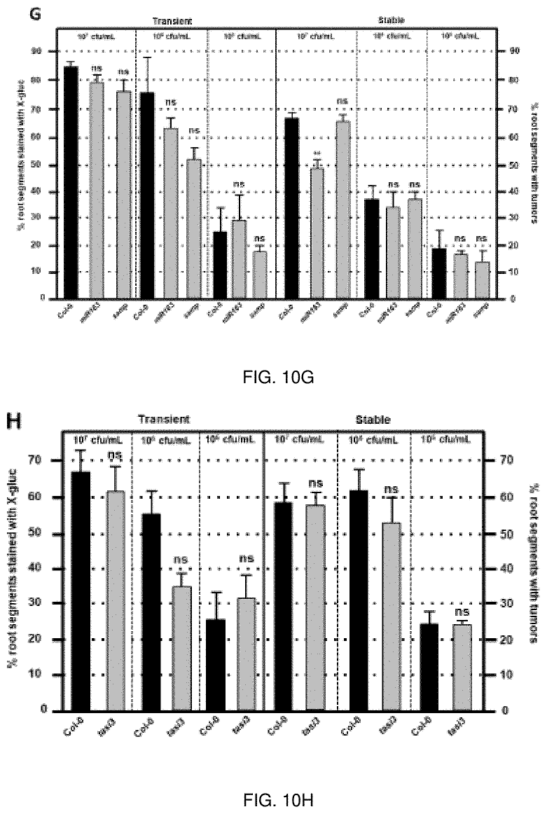

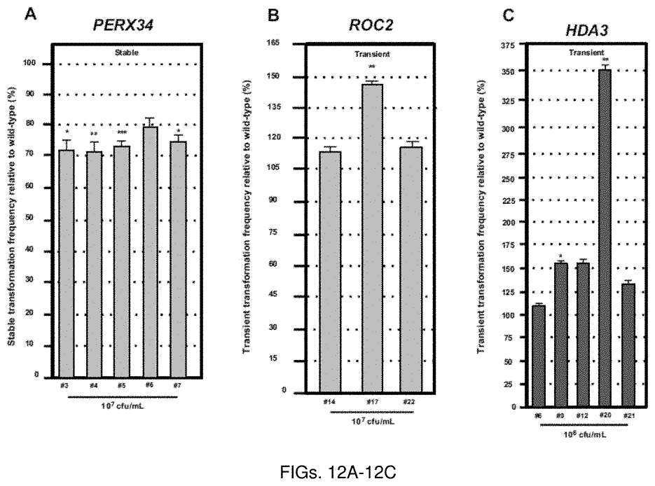

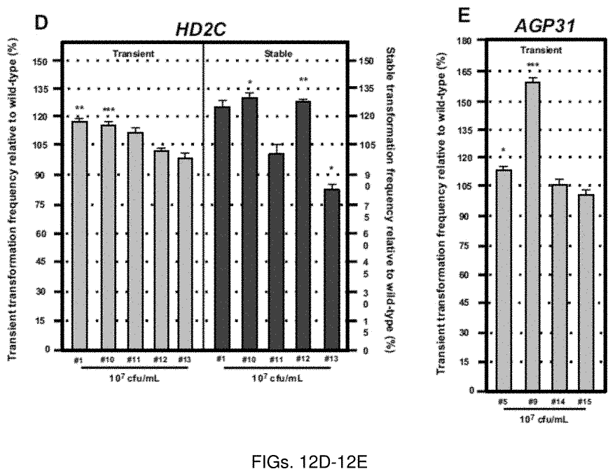

Induction of VirE2 increased transcript and protein levels of some arabinogalactan protein (AGP) genes. Arabinogalactan protein 17 (AGP17) was previously shown to be important for transformation by enhancing attachment of Agrobacterium to plant cells (Nam et al., 1999; Gaspar et al., 2004). A knockout mutant of the AGP14 gene was assayed for transformation susceptibility, and no significant difference in transformation was observed as compared to wild-type plants (FIG. 10E). Schlutz et al. (2002) identified 50 Arabidopsis genes encoding AGPs, and it is plausible that many have redundant functions in the plant cell. AGP31 showed increased protein levels (although at a p-value=0.27 by iBAQ analysis) in the presence of VirE2 (Table 3) and plants overexpressing AGP31 exhibited increased transient transformation susceptibility (Table 5; FIG. 12E). Therefore, VirE2 modulates both the transcript and protein levels of some AGPs to facilitate transformation.

Some heat shock protein transcript and protein levels increased in response to VirE2 induction, including the transcript encoding Heat Shock Protein 90 (HSP90). Park et al. (2014) demonstrated that overexpression of HSP90 increased Arabidopsis root transformation susceptibility and that HSP90 could act as a molecular chaperone to stabilize VirE2 and other proteins important for transformation. Upregulation of HSP90 by VirE2 could also facilitate transformation.

Histones, histone modifying enzymes, and cyclophilins showed increased protein levels in response to VirE2 (Table 4) and have previously been shown to play important roles in transformation (Deng et al., 1998; Nam et al., 1999; Bako et al., 2003; Crane and Gelvin, 2007; Tenea et al., 2009). Histone H2A2 (HTA2) and histone H4 (HIS4; formerly HFO4) protein levels increased three hours after induced VirE2 expression (Table 4). Overexpression of HIS4, HTA2, and some other histone H2A variants increased transformation susceptibility of Arabidopsis (Tenea et al., 2009). The histone deacetylases HD2C (formerly HDT3) and HDA3 (formerly HDT1) also showed increased protein levels in response to VirE2 (Table 4). Crane and Gelvin (2007) showed that RNAi-mediated silencing of HDA3 and other chromatin-related genes resulted in reduced transformation and T-DNA integration. Plants overexpressing HDA3 had enhanced transient transformation susceptibility (Table 5; FIG. 12C), whereas HD2C overexpressing plants had increased transient and stable transformation rates compared to wild-type plants (Table 5; Supplemental FIG. 6D). Increased levels of these histones and histone modifying proteins in response to VirE2 may also facilitate transformation.

VirD2 interacts with various cyclophilin proteins, and this interaction is important for efficient transformation (Deng et al., 1998; Bako et al., 2003). Two cyclophilin proteins, ROC2 and ROCS, showed increased protein levels post-VirE2 induction (Table 4). The studies presented herein also show that plants overexpressing ROC2 have increased transformation susceptibility (Table 5; FIG. 12B). Taken together, the present disclosure provides data suggest that VirE2 increases the levels of some cyclophilin proteins, facilitating transformation.

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### Example 1

### Methods & Materials

### Plasmid and Strain Constructions

Table 7 lists the plasmids and strains used in this study. To make a cloning vector with an inducible promoter (Pi), a blunted SphI-XhoI fragment containing the LexA operator and a minimal CaMV 35S promoter from pER8 (Zuo et al., 2000) was ligated to the blunted AgeI-XhoI plasmid pE3542 to make pE4224.

TABLE 7
Bacterial strains used in this study

| Strain | | Antibiotic | Reference or |
|---|---|---|---|
| name | Description | resistance^{a} | source |
| | E. coli strains | | |
| DH10B | F⁻ mcrA Δ (mrr-hsdRMS-mcrBC) | None | Durfee et al., |
| | Φ80dlacZΔM15 ΔlacX74 endA1 recA1 | | 2008 |
| | deoR Δ (ara, leu)7697 araD139 galU | | |
| | galK nupG rpsL λ⁻ | | |
| TOP10 | F− mcrA Δ (mrr-hsdRMS-mcrBC) | None | Invitrogen |
| | Φ80lacZΔM15 Δ lacX74 recA1 araD139 | | |
| | Δ (araleu)7697 galU galK rpsL | | |
| | (StrR) endA1 nupG | | |
| Stable | F′ proA + B + lacl^{q} Δ (lacZ)M15 zzf::Tn10 | None | New England |
| | (TetR) Δ (ara-leu) 7697 araD139 fhuA | | Biolabs |
| | ΔlacX74 galK16 galE15 e14- | | |
| | Φ80dlacZΔM15 recA1 relA1 endA1 | | |
| | nupG rpsL (StrR) rph spoT1 | | |
| | Δ (mrr-hsdRMS-mcrBC) | | |
| E886 | pBluescript (pBS) II KS (+) in DH5α | Amp | Stratagene |
| E3542 | pSAT1-Venus-C | Amp | Lee et al., |
| | | | 2008 |
| E3561 | pSAT1-P_{35S}-Venus-VirD2 | Amp | Lee et al., |
| | | | 2008 |
| E3759 | pSAT6-VirE2-Venus | Amp | Lee et al., |
| | | | 2008 |
| E4145 | pPZP-RCS2-P_{ocs}-hptll-R1 | Spec | Lee et al., |
| | | | 2012; |
| | | | This study |
| E4215 | T-DNA binary vector XVE-hptll | Spec | Lapham et al., |
| | | | 2018 |
| E4223 | T-DNA binary vector XVE-Pₙₒₛ- | Spec | This study |
| | mCherry-ABD2-hptll | | |
| E4224 | pSAT1-Inducible Promoter (minimal | Amp | Lapham et al., |
| | 355-LexA operator; pl) | | 2018; |
| | | | This study |
| E4229 | pSAT5-P_{35S}-VirE2 | Amp | Lee et al., |
| | | | 2008 |
| E4276 | pSAT1-pl-VirE2 | Amp | This study |
| E4282 | pSAT1-pl-VirE2-Venus | Amp | This study |
| E4288 | T-DNA binary vector XVE-inducible | Spec | Lapham et al., |
| | VIP1 | | 2018; |
| | | | This study |
| E4289 | T-DNA binary vector XVE-inducible | Spec | This study |
| | VirE2 | | |
| E4292 | T-DNA binary vector XVE-inducible | Spec | This study |
| | VirE2-Venus-Pₙₒₛ-MCherry-ABD2-hptll | | |
| E4297 | pSAT1A-P35s-Multi-cloning Site (MCS)- | Amp | Lee et al., |
| | T_{35S} | | 2008 |
| E4372 | pSAT5-P_{35S}-mCherry-ABD2 | Amp | This study |
| E4373 | pSAT4-Pₙₒₛ-Cerulean-VirD2NLS | Amp | Lee et al., |
| | | | 2008 |
| E4375 | pSAT4-Pₙₒₛ-Cerulean-SV40NLS | Amp | Lee et al., |
| | | | 2008 |
| E4376 | T-DNA binary vector XVE-inducible | Spec | This study |
| | VirE2-Venus-Pₙₒₛ-MCherry-ABD2-hptll- | | |
| | Pₙₒₛ-Cerulean-SV40NLS | | |
| E4377 | T-DNA binary vector XVE-inducible | Spec | This study |
| | VirE2-Venus-hptll-Pₙₒₛ-Cerulean- | | |
| | SV40NLS | | |
| E4380 | T-DNA binary vector XVE-inducible | Spec | This study |
| | VirE2-Venus-P_{35S}-mCherry-ABD2- | | |
| | hpt11-Pnos-Cerulean-SV40NLS | | |
| E4386 | T-DNA binary vector XVE-inducible | Spec | This study |
| | VirE2-Venus-P_{35S}-mCherry-ABD2-hptll | | |
| E4389 | T-DNA binary vector XVE-inducible | Spec | This study |
| | VirE2-Venus-P_{35S}-mCherry-ABD2- | | |
| | hptll-Pₙₒₛ-Cerulean-VirD2NLS | | |
| E4433 | pSAT1-P_{35S}-Venus-VirD2NLS | Amp | This study |
| E4434 | pSAT6-P_{35S}-VirE2-Venus-VirD2NLS | Amp | This study |
| E4435 | T-DNA binary vector XVE-inducible | Spec | This study |
| | VirE2-Venus-VirD2NLS-P_{35S}-mCherry- | | |
| | ABD2-hptll-Pₙₒₛ-Cerulean-VirD2NLS | | |
| E4436 | pSAT1-pl-VirE2-Venus-VirD2NLS | Amp | This study |
| E4438 | T-DNA binary vector XVE-inducible | Spec | This study |
| | VirE2-Venus-hptll-Pₙₒₛ-Cerulean- | | |
| | VirD2NLS | | |
| E4439 | T-DNA binary vector XVE-inducible | Spec | This study |
| | VirE2-Venus-VirD2NLS-hptll-Pₙₒₛ- | | |
| | Cerulean-VirD2NLS | | |
| E4515 | pSAT1-P_{35S}-MCS-T_{35S} | Kan | Lee et al., |
| | | | 2008; |
| | | | This study |
| E4594 | PERX34: DKLAT3G49120 cDNA clone | Spec | ABRC* |
| E4597 | AGP31: DKLAT1G28290 cDNA clone | Spec | ABRC* |
| E4601 | HDA3: DKLAT3G44750 cDNA clone | Spec | ABRC* |
| E4602 | HD2C: DKLAT5G03740 cDNA clone | Spec | ABRC* |
| E4603 | ROC2: DKLAT3G56070 cDNA clone | Spec | ABRC* |
| E4622 | pSAT1A-P_{35S}-PERX34-T_{35S} | Amp | This study |
| E4623 | pPZP-P_{35S}-PERX34-T_{35S}-P_{ocs}-hptll-RI | Spec | This study |
| E4626 | pBS-AGP31 | Amp | This study |
| E4627 | pSAT1A-P_{35S}-AGP3/-T_{35S} | Amp | This study |
| E4628 | pPZP-P_{35S}-AGP3/-T_{35S}-P_{ocs}-hptll-RI | Spec | This study |
| E4629 | pBS-HDA3 | Amp | This study |
| E4630 | pSAT1-P_{35S}-HDA3-T_{35S} | Kan | This study |
| E4631 | pPZP-P_{35S}-HDA3-T_{35S}-P_{ocs}-hptll-RI | Spec | This study |
| E4633 | pBS-HD2C | Amp | This study |
| E4634 | pSAT1A-P_{35S}-HD2C-T_{35S} | Amp | This study |
| E4635 | pPZP-P_{35S}-HD2C-T_{35S}-P_{ocs}-hptll-RI | Spec | This study |
| E4637 | pBS-ROC2 | Amp | This study |
| E4638 | pSAT1-P_{35S}-ROC2-T_{35S} | Kan | This study |
| E4639 | pPZP-P_{35S}-ROC2-T_{35S}-P_{ocs}-hptll-RI | Spec | This study |
| Strain | | Antibiotic | |
| name | Description | resistance^{a} | Reference |
| | Agrobacterium strains | | |
| A208 | Tumorigenic; pTiT37 in A136 | Rif | Sciaky et al., |
| | | | 1978 |
| EHA105 | Non-tumorigenic, disarmed pTiBO542 | Rif | Hood et al., |
| | without Kan gene in A136 | | 1993 |
| GV3101 | Non-tumorigenic, disarmed pTiC58 in | Rif, Gent | Koncz and |
| | C58 background | | Schell, |
| | | | 1986 |
| At2 | Non-tumorigenic; A136 | Rif | Sciaky et al., |
| | | | 1978 |
| At849 | pBISN1 in GV3101 | Rif, Gent, | Narasimhulu et al., |
| | | Kan | 1996 |
| At1529 | pBISN1 in EHA105 | Rif, Kan | This study |
| At1879 | pBISN2 in EHA105 with in-frame | Rif, Kan, | This study |
| | deletion of virE2 | Spec | |
| At2082 | pE4288 in GV3101 | Rif, Gent, | Lapham et al., |
| | | Spec | 2018 |
| At2091 | pE4289 in GV3101 | Rif, Gent, | This study |
| | | Spec | |
| At2155 | pE4438 in GV3101 | Rif, Gent, | This study |
| | | Spec | |
| At2156 | pE4439 in GV3101 | Rif, Gent, | This study |
| | | Spec | |
| At2259 | pE4623 in GV3101 | Rif, Gent, | This study |
| | | Spec | |
| At2264 | pE4628 in GV3101 | Rif, Gent, | This study |
| | | Spec | |
| At2265 | pE4631 in GV3101 | Rif, Gent, | This study |
| | | Spec | |
| At2267 | pE4635 in GV3101 | Rif, Gent, | This study |
| | | Spec | |
| At2268 | pE4639 in GV3101 | Rif, Gent, | This study |
| | | Spec | |

^{a}Amp, ampicillin; Gent, gentamicin; Kan, kanamycin; Rif, rifampicin; Spec, spectinomycin;
*ABRC: Arabidopsis Biological Resource Center, The Ohio State University.

To make the pPi-VirE2-Venus construction, a SwaI-NotI fragment containing the VirE2-Venus fragment from pE3759 was cloned into the SwaI-NotI sites of pE4224 to make Pi-VirE2-Venus (pE4282). The AscI fragment from pE4282 containing the expression cassette pPi-VirE2-Venus and an I-SceI fragment containing Pₙₒₛ-Cerulean-NLS from pE4373 were cloned into the AscI and I-SceI sites, respectively, of a binary vector derived from pE4215 containing an XVE expression cassette to make pE4438 (pPZP-Pi-VirE2-Venus-Pₙₒₛ-Cerulean-NLS).

To make the pPi-VirE2-Venus-NLS construction, pSAT1-P35s-Venus-VirD2 (pE3561) was digested with HindIII before self-ligating the backbone fragment to create pSAT1-P35s-Venus-NLS (pE4433). A PstI-NotI fragment from pE4433 was used to replace the PstI-NotI fragment of pE3759 to make pE4434 (pSAT6-P₃₅s-VirE2-Venus-NLS). A SwaI-NotI fragment from pE4434 was cloned into the SmaI-NotI sites of pE4224 to make pE4436 (pSAT1-Pi-VirE2-Venus-NLS). An AscI fragment containing the Pi-VirE2-Venus-NLS expression cassette from pE4436 was cloned into the AscI site (to replace the Pi-VirE2-Venus expression cassette) of pE4389 to make pE4435. pE4435 was digested with I-CeuI and self-ligated to make pE4439 (pPZP-Pi-VirE2-Venus-NLS-Pₙₒₛ-Cerulean-NLS). pE4438 and pE4439 were separately introduced into A. tumefaciens GV3101 (Van Larebeke et al., 1974) by electroporation to make A. tumefaciens At2155 and At2156, respectively.

To generate a binary vector carrying the Pi-VirE2 expression cassette, a SwaI-NotI fragment containing the VirE2 gene from pE4229 was cloned into the SmaI-NotI sites of pE4224 to create pE4276. The AscI fragment containing pPi-VirE2 was cloned into the AscI sites of pE4215 to generate pE4289. pE4289 was electroporated into A. tumefaciens GV3101 to make A. tumefaciens At2091.

To generate the constitutive overexpression constructs for proteins whose levels are increased in the presence of VirE2, cDNA clones were ordered from the Arabidopsis Biological Resource Center (ABRC) for each selected gene (Table 7). Each gene was amplified from the cDNA clone using PCR and primers with flanking sequences containing restriction enzyme sites (Table 8). Either Phusion High-Fidelity DNA Polymerase (New England Biolabs) or Platinum SuperFi DNA Polymerase (Invitrogen) was used and the reactions were conducted according to the manufacturers' protocols. The PCR fragments containing PERX34 (At3g49120) were digested with restriction enzymes which recognized their flanking sequences (Table 8) before cloning those fragments into the same sites on pE4297 to create pE4622 (Table 7). The blunt-end PCR fragments containing AGP31 (At1g28290), HDA3 (At3g44750), HD2C (At5g03740), and ROC2 (At3g56070) were cloned into pBluescript KS₊ cut with EcoRV to make pE4626, pE4629, pE4633, and pE4637 respectively (Tables 7 & 8). These plasmids were also sequenced. The EcoRI-BamHI fragments from pE4629 (HDA3) and pE4637 (ROC2) were cloned into the same sites of pE4515 to make pE4630 and pE4638, respectively. The SalI-BamHI fragment from pE4626 (AGP31) and the BgIII-BamHI fragment from pE4633 (HD2C) were cloned into the same sites of pE4297 to make pE4627 and pE4634, respectively. The AscI fragments containing the overexpression cassettes from pE4622 (PERX34), pE4627 (AGP31), pE4630 (HDA3), pE4634 (HD2C), and pE4638 (ROC2) were cloned into the AscI site of the binary vector pE4145 to make pE4623, pE4628, pE4631, pE4635, and pE4639, respectively. Each binary vector was electroporated into A. tumefaciens GV3101 to make A. tumefaciens strains At2259, At2264, At2265, At2267, and At2268, respectively.

TABLE 8
Primer sequences used in this study

| | | SEQ | | |
|---|---|---|---|---|
| Primer | Sequence | ID | Tm | |
| Name | (5′ to 3′) | NO: | (° C.) | Purpose |
| VirE2 | CTTGG | 1 | 58 | RT-qPCR |
| qPCR | TGAAG | | | |
| Fwd | CAGCT | | | |
| | GACAA | | | |
| | ATACT | | | |
| | C | | | |
| Universal | AGACT | 2 | 58.6 | RT-qPCR |
| qPCR | GGTGA | | | |
| Rev | TTTTT | | | |
| | GCGGA | | | |
| | CTCTA | | | |
| | G | | | |
| ADH1 | CGGGG | 3 | 58.2 | RT-qPCR |
| (At1G771 | TTGTG | | | |
| 20) qPCR | GAAAA | | | |
| Fwd | GTACA | | | |
| | TGAAC | | | |
| A | | | | |
| DH1 | GCTTC | 4 | 59 | RT-qPCR |
| (At1G771 | AAGCA | | | |
| 20) qPCR | CCCAT | | | |
| Rev | GGTGA | | | |
| | TG | | | |
| PRKP | TGACC | 5 | 58.8 | RT-qPCR |
| (At1G518 | CGAAC | | | |
| 40) qPCR | TTCGA | | | |
| Fwd | CCTTT | | | |
| | ACC | | | |
| PRKP | TCAAT | 6 | 58.6 | RT-qPCR |
| (At1G518 | GAACC | | | |
| 40) qPCR | GCTTT | | | |
| Rev | GAGTA | | | |
| | GCGTA | | | |
| | TAC | | | |
| TAS4 | AAGTC | 7 | 59.1 | RT-qPCR |
| (At3G257 | ACTCA | | | |
| 95) qPCR | AACAC | | | |
| Fwd | TGACG | | | |
| | TGAAC | | | |
| | C | | | |
| TAS4 | CGTCC | 8 | 60.6 | RT-qPCR |
| (At3G257 | TTCAC | | | |
| 95) qPCR | CACGG | | | |
| Rev | CAATT | | | |
| | TCATG | | | |
| PR | CACTA | 9 | 58.3 | RT-qPCR |
| (AtT4G33 | TACTC | | | |
| 720) | AGGTT | | | |
| qPCR | GTGTG | | | |
| Fwd | GAGAA | | | |
| | ACTC | | | |
| PR | CCACT | 10 | 58.3 | RT-qPCR |
| (At4G337 | CGCCA | | | |
| 20) qPCR | ACCCA | | | |
| Rev | GTTAC | | | |
| LSU1 | GAGCT | 11 | 58.5 | RT-qPCR |
| (At3G495 | GGAGG | | | |
| 80) qPCR | TCGAG | | | |
| Fwd | TCTTT | | | |
| | AGAAC | | | |
| LSU1 | CTTAT | 12 | 57.7 | RT-qPCR |
| (At3G495 | TCTAC | | | |
| 80) qPCR | GAGGA | | | |
| Rev | AGAGA | | | |
| | CGACA | | | |
| | GAAG | | | |
| LRRPK | TCCTT | 13 | 59.7 | RT-qPCR |
| (At1G518 | CATCA | | | |
| 30) qPCR | GCTAG | | | |
| Fwd | AAGAC | | | |
| | CGAAC | | | |
| | ATG | | | |
| LRRPK | CCGAG | 14 | 60.6 | RT-qPCR |
| (At1G518 | CCAAT | | | |
| 30) qPCR | GGGGT | | | |
| Rev | CACTT | | | |
| | C | | | |
| AGP21 | AAAGA | 15 | 55 | RT-qPCR |
| (At1G553 | TCTAT | | | |
| 30) | GGAGG | | | |
| Geno Fwd | CAATG | | | |
| | AAGAT | | | |
| | G | | | |
| AGP21 | TTCTT | 16 | 56 | RT-qPCR |
| (At1G553 | AAGTC | | | |
| 30) | AAAAG | | | |
| Geno Rev | ATGAA | | | |
| | ACCAG | | | |
| | ATGC | | | |
| AtNTR2.6 | GAAGA | 17 | 59 | RT-qPCR |
| (At3G450 | GCATT | | | |
| 60) qPCR | ACTAT | | | |
| Fwd | GGAGC | | | |
| | GGAAT | | | |
| | GG | | | |
| AtNTR2.6 | CTTCA | 18 | 58.4 | RT-qPCR |
| (At3G450 | CTAGA | | | |
| 60) qPCR | CATGA | | | |
| Rev | GCCGG | | | |
| | AGATC | | | |
| FRO2 | CTGCA | 19 | 57.7 | RT-qPCR |
| (At1G015 | TTTTG | | | |
| 80) | GAGAA | | | |
| qFwd | AGACC | | | |
| | TAATC | | | |
| | TCAAG | | | |
| FR02 | AGAGT | 20 | 58.5 | RT-qPCR |
| (At1 G015 | TATAT | | | |
| 80) | ACGCA | | | |
| qRev | ATCAC | | | |
| | CAGCT | | | |
| | GAAAC | | | |
| TMP | GAGTC | 21 | 57.5 | RT-qPCR |
| (At4G372 | GTCCG | | | |
| 90) qFwd | CTTGG | | | |
| | TCTAA | | | |
| | C | | | |
| TMP | CTTGG | 22 | 58 | RT-qPCR |
| (At4G372 | ACCTG | | | |
| 90) qRev | AGTGC | | | |
| | TTAAC | | | |
| | AAATC | | | |
| | G | | | |
| HSP90 | GCTAG | 23 | 57.6 | RT-qPCR |
| (At5G526 | GATTC | | | |
| 40) | ACAGG | | | |
| qFwd | ATGTT | | | |
| | GAAGT | | | |
| | TG | | | |
| HSP90 | ACTTC | 24 | 58.1 | RT-qPCR |
| (At5G526 | CTCCA | | | |
| 40) qRev | TCTTG | | | |
| | CTCTC | | | |
| | TTCAG | | | |
| LEA4-5 | GTCGG | 25 | 58.2 | RT-qPCR |
| (At5G067 | ACAAC | | | |
| 60) qFwd | CGCTC | | | |
| | ATAAC | | | |
| | AC | | | |
| LEA4-5 | AGAAC | 26 | 57.6 | RT-qPCR |
| (At5G067 | AAGTG | | | |
| 60) qRev | AACAA | | | |
| | CACCG | | | |
| | TTTAT | | | |
| | CC | | | |
| CBFP | ACAAG | 27 | 58.3 | RT-qPCR |
| (AT5G570 | TCAAC | | | |
| 10)qFwd | CTTTC | | | |
| | TCCTC | | | |
| | GTGTA | | | |
| | G | | | |
| CBFP | GCTTG | 28 | 57.9 | RT-qPCR |
| (At5G570 | GAAGA | | | |
| 10) qRev | CCCAT | | | |
| | GCAAG | | | |
| | ATAG | | | |
| Left | TGGTT | 29 | 61.5 | T-DNA |
| Border | CACGT | | | insertion |
| Primer | AGTGG | | | line |
| (SALK) | GCCAT | | | genotyping |
| | CG | | | |
| 12965 | AAGAG | 30 | 58 | T-DNA |
| IncRNA | CTCCT | | | insertion |
| Geno Fwd | AGCTA | | | line |
| (SALK_08 | TATAT | | | genotyping |
| 6573) | TCTGG | | | |
| | AGACT | | | |
| | C | | | |
| 12965 | TTCCG | 31 | 59.6 | T-DNA |
| IncRNA | CGGGA | | | insertion |
| Geno Rev | TTAAC | | | line |
| (SALK_08 | TGTTA | | | genotyping |
| 6573) | AAAGA | | | |
| | TTCAA | | | |
| | AAAC | | | |
| AtPSK3 | ATGTG | 32 | 55.6 | T-DNA |
| LP | TTACG | | | insertion |
| (SALK_04 | CAGTT | | | line |
| 4781) | TCGTC | | | genotyping |
| | C | | | |
| AtPSK3 | AGCTT | 33 | 53.9 | T-DNA |
| RP | TGCTT | | | insertion |
| (SALK_04 | CATGT | | | line |
| 4781) | TCTTG | | | genotyping |
| | G | | | |
| ACS6 | AAAGA | 34 | 58 | T-DNA |
| Geno Fwd | TCTAT | | | insertion |
| (SALK_05 | GGTGG | | | line |
| 4467) | CTTTT | | | genotyping |
| | GCAAC | | | |
| | AG | | | |
| ACS6 | TTCTT | 35 | 57.9 | T-DNA |
| Geno Rev | AAGTT | | | insertion |
| (SALK_05 | AAGTC | | | line |
| 4467) | TGTGC | | | genotyping |
| | ACGGA | | | |
| | CTAG | | | |
| TST18 | AAAGA | 36 | 56.1 | T-DNA |
| Geno Fwd | TCTAT | | | insertion |
| (CS86728 | GTCTC | | | line |
| 5) | AATCA | | | genotyping |
| | ATCTC | | | |
| | CTCC | | | |
| TST18 | TTCTT | 37 | 56.5 | T-DNA |
| Geno Rev | AAGTT | | | insertion |
| (CS86728 | AATTA | | | line |
| 5) | GCAGA | | | genotyping |
| | TGGCT | | | |
| | CCTC | | | |
| PR5 LP | CATTT | 38 | 52.1 | T-DNA |
| (SALK_05 | CATTA | | | insertion |
| 5063C) | ATGGC | | | line |
| | TCGCT | | | genotyping |
| | C | | | |
| PR5 RP | ATTGC | 39 | 55.7 | T-DNA |
| (SALK_05 | TGTTA | | | insertion |
| 5063C) | TGGCC | | | line |
| | ACAGA | | | genotyping |
| | C | | | |
| AGP14 | TTTAG | 40 | 55.1 | T-DNA |
| LP | GAGTT | | | insertion |
| (SALK_09 | GTGCC | | | line |
| 6806) | CATGT | | | genotyping |
| | C | | | |
| AGP14 | CCTTA | 41 | 52.4 | T-DNA |
| RP | ACGTG | | | insertion |
| (SALK_09 | TCATA | | | line |
| 6806) | AATCA | | | genotyping |
| | ATTCC | | | |
| tasi4 LP | CGAGG | 42 | 51.7 | T-DNA |
| (SALK_06 | TTAAA | | | insertion |
| 6997) | ATTCC | | | line |
| | GAAAG | | | genotyping |
| | G | | | |
| tasi4 RP | GTCCG | 43 | 54 | T-DNA |
| (SALK_06 | CAATA | | | insertion |
| 6997) | CGTAA | | | line |
| | AACTC | | | genotyping |
| | G | | | |
| miR163 | ACCCG | 44 | 57 | T-DNA |
| LP Geno | GTGGA | | | insertion |
| (CS87979 | TAAAA | | | line |
| 7) | TCGAG | | | genotyping |
| | TTC | | | |
| miR163 | TCAAG | 45 | 57 | T-DNA |
| RP | CGTCC | | | insertion |
| (CS87979 | AGACT | | | line |
| 7) | TCAGA | | | genotyping |
| | TTG | | | |
| SAMP LP | TGTTG | 46 | 54 | T-DNA |
| (SALK_20 | CATTT | | | insertion |
| 9995C) | GTGGA | | | line |
| | CAAGA | | | genotyping |
| | C | | | |
| SAMP RP | TGGAG | 47 | 56.1 | T-DNA |
| (SALK_20 | TGATC | | | insertion |
| 9995C) | TCGTA | | | line |
| | ACGGA | | | genotyping |
| | C | | | |
| TAS3 | TGAGA | 48 | 52.9 | T-DNA |
| RP2 | AGAGA | | | insertion |
| (N432182 | GCAAA | | | line |
| GABI-Kat) | GAAAC | | | genotyping |
| | TTC | | | |
| TAS3LP2 | CATGT | 49 | 52.6 | T-DNA |
| (N432182 | GGAAA | | | insertion |
| GABI-Kat) | CAAAC | | | line |
| | GTATG | | | genotyping |
| | AAG | | | |
| GABI-Kat | ATAAT | 50 | 56.9 | T-DNA |
| T-DNA | AACGC | | | insertion |
| primer | TGCGG | | | line |
| 8474 | ACATC | | | genotyping |
| | TACAT | | | |
| | TTT | | | |
| EXL1 | TCTAT | 51 | 55.4 | T-DNA |
| Geno Fwd | TACAT | | | insertion |
| (SALK_01 | TCGCG | | | line |
| 0243C) | GCAAT | | | genotyping |
| | ATTCG | | | |
| EXL1 | GCTAT | 52 | 56.5 | T-DNA |
| Geno Rev | ACGTG | | | insertion |
| (SALK_01 | TAGGG | | | line |
| 0243C) | CTCAT | | | genotyping |
| | AAGAC | | | |
| MEE39 | ATGAA | 53 | 56.4 | T-DNA |
| Geno Fwd | GAATC | | | insertion |
| (SALK_06 | TTTGT | | | line |
| 5070C) | TGGGT | | | genotyping |
| | TTTTC | | | |
| | TGTC | | | |
| MEE39 | GAACG | 54 | 55.8 | T-DNA |
| Geno Rev | ATCAT | | | insertion |
| (SALK_06 | AAACA | | | line |
| 5070C) | TCTTT | | | genotyping |
| | CGGGT | | | |
| | AC | | | |
| RBC3B | AAAGA | 55 | 64.3 | T-DNA |
| Geno Fwd | TCTAT | | | insertion |
| (SALK_11 | GGCTT | | | line |
| 7835) | CCTCT | | | genotyping |
| | ATGCT | | | |
| | CTCC | | | |
| | TCCGC | | | |
| RBC3B | TTGGT | 56 | 65 | T-DNA |
| Geno Rev | ACCAA | | | insertion |
| (SALK_11 | GAAAT | | | line |
| 7835) | TAAGC | | | genotyping |
| | TTCGG | | | |
| | TGAAG | | | |
| | CTTGG | | | |
| | GG | | | |
| ABAH3 | AAGAG | 57 | 59.1 | T-DNA |
| Geno Fwd | CTCAT | | | insertion |
| (SALK_07 | GGATT | | | line |
| 8170) | TCTCC | | | genotyping |
| | GGTTT | | | |
| | G | | | |
| ABAH3 | TTGGT | 58 | 60.4 | T-DNA |
| Geno Rev | ACCCT | | | insertion |
| (SALK_07 | ATGGT | | | line |
| 8170) | TTTCG | | | genotyping |
| | TTCCA | | | |
| | AGG | | | |
| NRT2.6 | CACCA | 59 | 55.7 | T-DNA |
| LP | AAGAG | | | insertion |
| (SALK_20 | AGCTC | | | line |
| 4101C) | CACAA | | | genotyping |
| | G | | | |
| NRT2.6 | GGCTC | 60 | 55.2 | T-DNA |
| RP | TATTG | | | insertion |
| (SALK_20 | GAACC | | | line |
| 4101C) | TCCTT | | | genotyping |
| | G | | | |
| CUP LP | CATCG | 61 | 53.9 | T-DNA |
| (SALK_20 | TCACC | | | insertion |
| 1444C) | ACAAT | | | line |
| | CTTTC | | | genotyping |
| | C | | | |
| CUP RP | GGACA | 62 | 52.8 | T-DNA |
| (SALK_20 | AAAGT | | | insertion |
| 1444C) | TTGCA | | | line |
| | TATGG | | | genotyping |
| | C | | | |
| AtNTR2.1 | GTTGG | 63 | 60.1 | T-DNA |
| Geno Fwd | TTGCA | | | insertion |
| (SALK_03 | CATCA | | | line |
| 5429C) | TCATG | | | genotyping |
| | GGAAT | | | |
| | CTTG | | | |
| AtNTR2.1 | GGCGT | 64 | 60.4 | T-DNA |
| qPCR | CCACC | | | insertion |
| Rev | CTCTG | | | line |
| (SALK_03 | ACTTG | | | genotyping |
| 5429C) | | | | |
| OEP6 | AAAGA | 65 | 57.5 | T-DNA |
| Geno Fwd | TCTAT | | | insertion |
| (CS86277 | GGTGG | | | line |
| 4) | AGAAG | | | genotyping |
| | TCAGG | | | |
| | AG | | | |
| OEP6 | TCCTT | 66 | 57.6 | T-DNA |
| Geno Rev | AAGAT | | | insertion |
| (CS86277 | TCTCA | | | line |
| 4) | CTCAC | | | genotyping |
| | CATAT | | | |
| | TCAGG | | | |
| ESM1 LP | TGAAC | 67 | 55.2 | T-DNA |
| (SALK_15 | GTCTG | | | insertion |
| 0833C) | TGAAG | | | line |
| | TTCAC | | | genotyping |
| | G | | | |
| ESM1 RP | TGCCG | 68 | 53.6 | T-DNA |
| (SALK_15 | GTTTT | | | insertion |
| 0833C) | GTATT | | | line |
| | CTTGT | | | genotyping |
| | C | | | |
| RLD17 LP | CAAGA | 69 | 54.3 | T-DNA |
| (SALK_11 | GCTGA | | | insertion |
| 5776C) | AAGCC | | | line |
| | TCAAA | | | genotyping |
| | C | | | |
| RLD17 | TTACC | 70 | 53.7 | T-DNA |
| RP | AGGAT | | | insertion |
| (SALK_11 | GAGAT | | | line |
| 5776C) | GATCG | | | genotyping |
| | G | | | |
| PP2C LP | CACCA | 71 | 58.7 | T-DNA |
| (SALK_10 | ATCTT | | | insertion |
| 4445) | CATGG | | | line |
| | AGATC | | | genotyping |
| | G | | | |
| PP2C RP | GATTA | 72 | 52.4 | T-DNA |
| (SALK_10 | ATTTC | | | insertion |
| 4445) | GGCCA | | | line |
| | ATGCT | | | genotyping |
| | C | | | |
| ADH1 LP | CGATG | 73 | 55.1 | T-DNA |
| (SALK_05 | GGTAC | | | insertion |
| 2699) | ACCGA | | | line |
| | TTACT | | | genotyping |
| | G | | | |
| ADH1 RP | AAAGA | 74 | 53.4 | T-DNA |
| (SALK_05 | TCGGC | | | insertion |
| 2699) | AACAC | | | line |
| | ATGAT | | | genotyping |
| | C | | | |
| PERCB/ | AAGAA | 75 | 55.9 | Cloning of |
| 34 | TTCAT | | | over- |
| (At3G491 | GCATT | | | expression |
| 20)-OE- | TCTCT | | | lines |
| EcoRI- | TCGTC | | | |
| Fwd | TTC | | | |
| PERCB/ | AAGGA | 76 | 57.8 | Cloning of |
| 34 | TCCTC | | | over- |
| (At3G491 | ACATA | | | expression |
| 20)-OE- | GAGCT | | | lines |
| BamHI- | AACAA | | | |
| Rev | AGTC | | | |
| AGP31 | AAAGA | 77 | 55 | Cloning of |
| (At1G282 | TCTAT | | | over- |
| 90)-OE- | GGGTT | | | expression |
| BgIII- | TCATT | | | lines |
| Fwd | GGTAA | | | |
| | GAG | | | |
| AGP31 | AAGGA | 78 | 59.3 | Cloning of |
| (AHG282 | TCCTC | | | over- |
| 90)-OE- | ATTTG | | | expression |
| BamHI- | GGGCA | | | lines |
| Rev | AGAC | | | |
| HDT1/HD | AAGAA | 79 | 57.3 | Cloning of |
| A3 | TTCAT | | | over- |
| (At3G447 | GGAGT | | | expression |
| 50)-OE- | TCTGG | | | lines |
| EcoRI- | GGAAT | | | |
| Fwd | TG | | | |
| HDT1/HD | AAGGA | 80 | 61.7 | Cloning of |
| A3 | TCCTC | | | over- |
| (At3G447 | ACTTG | | | expression |
| 50)-OE- | GCAGC | | | lines |
| BamHI- | AGC | | | |
| Rev | | | | |
| HDT3/HD | AAAGA | 81 | 56.2 | Cloning of |
| 2C | TCTAT | | | over- |
| (At5G037 | GGAGT | | | expression |
| 40)-OE- | TCTGG | | | lines |
| BgIII- | GGTG | | | |
| Fwd | | | | |
| HDT3/HD | AAGGA | 82 | 61.4 | Cloning of |
| 2C | TCCTC | | | over- |
| (At5G037 | AAGCA | | | expression |
| 40)-OE- | GCTGC | | | lines |
| BamHI- | ACTG | | | |
| Rev | | | | |
| ROC2 | AAGAA | 83 | 55.5 | Cloning of |
| (At3G560 | TTCAT | | | over- |
| 70)-OE- | GGCGA | | | expression |
| EcoRI- | ATCCT | | | lines |
| Fwd | AAAGT | | | |
| | C | | | |
| ROC2 | AAGGA | 84 | 58.3 | Cloning of |
| (At3G560 | TCCTT | | | over- |
| 70)-OE- | ATGAA | | | expression |
| BamHI- | CTTGG | | | lines |
| Rev | GTTCT | | | |
| | TGAG | | | |

Isolation and Transfection of Tobacco BY-2 Protoplasts. Protoplasts were isolated from tobacco BY-2 cells and transfected as described by Lee et al. (2012). A plasmid encoding a nuclear mRFP marker (pE3170) was co-transfected with the appropriate clones into the protoplasts. Imaging was performed 16 hours post-transfection using a Nikon A1R Confocal Laser Microscope System as described in Shi et al. (2014).

Generation and selection of inducible VirE2, VirE2-Venus, VirE2-Venus-NLS, VIP1, and transgenic A. thaliana plants constitutively overexpressing selected genes. Wild-type A. thaliana plants (ecotype Col-0) were individually transformed by A. tumefaciens At2155, At2156, At2091, At2259, At2264, At2265, At2267, or At2268 using a flower dip protocol (Clough and Bent, 1998). To generation seeds from the transformed plants were surface sterilized for 15-20 min in a 50% commercial bleach and 0.1% sodium dodecylsulfate (SDS) solution before washing five times with sterile water. After overnight incubation in water at 4° C., the seeds were plated on solidified Gamborg's B5 medium containing 100 mg/mL Timentin and 20 mg/mL hygromycin. The seeds were placed at 23° C. under a 16/8-hrs light/dark cycle. T1 generation hygromycin-resistant seedlings for the inducible lines were transplanted to soil and grown under the same temperature and light conditions. For inducible VirE2 plants, hygromycin was used to select for homozygous plants. Homozygous T2 plants containing the inducible VirE2-Venus and VirE2-Venus-NLS constructions were used for future experiments. T1 generation hygromycin-resistant seedlings for each of the constitutive overexpression lines were transferred to baby food jars containing solidified B5 medium for 10-14 days. Roots of each plant were cut into 3-5 mm segments and assayed as described in Tenea et al. (2009). Root segments were infected with A. tumefaciens At849 (GV3101::pMP90 (Koncz and Schell, 1986) containing pBISN1 (Narasimhulu et al., 1996) to measure transient transformation at a concentration of 10⁶ cfu/mL (Tables 7 & 8). Shoots were re-rooted in solidified B5 medium in the jars for 7 to 10 days before transferring plantlets to soil.

Transgenic plants overexpressing VIP1 were generated using A. tumefaciens At2082 as previously described (Lapham et al., 2018).

Imaging of VirE2-Venus and VirE2-Venus-NLS transgenic A. thaliana roots. Inducible VirE2-Venus and VirE2-Venus-NLS seedlings (T2 generation) were germinated on B5 medium containing 100 mg/mL Timentin and 20 mg/mL hygromycin. The seedlings were transferred after two weeks to plates containing B5 medium lacking antibiotics. These plates were placed vertically in racks to promote root growth on the surface of the medium. After 10 days, the plates were placed horizontally and B5 liquid medium containing 10 μM β-estradiol dissolved in DMSO (β-estradiol solution) or B5 plus DMSO only (control solution) was pipetted onto the surface until a thin layer covered the root tissue (4-5 mL). The roots were incubated in the solution for 9 hours before imaging using a Nikon A1R Confocal Laser Microscope System as described in Shi et al. (2014).

Assaying inducible VirE2-Venus and VirE2-Venus-NLS transgenic A. thaliana roots for complementation of virE2⁻ mutant Agrobacterium. Three transgenic lines of Inducible VirE2-Venus (Lines #4-6) and VirE2-Venus-NLS (Lines #4-6) seedlings (T2 generation) were grown and treated with either 10 μM β-estradiol induction or control solution for 24 h as described above. Root segments were infected as described in Tenea et al. (2009) using either A. tumefaciens At1529 or the virE2-mutant strain At1879 at a concentration of 10⁶ or 10⁸ cfu/mL, respectively (Table 7). Three replicates were assayed for each line with root segments pooled from 10-30 plants for each replicate. A total of 80 or more root segments were scored for each data point and statistical analysis was performed using ANOVA.

VirE2, VirE2-Venus, VirE2-Venus-NLS, and VIP1 Induction in the presence of Agrobacterium. Inducible VirE2 (line #10) or inducible VIP1 (line #12) T3 generation plants were grown and assayed as described above, except that A. tumefaciens A136 (lacking a Ti plasmid) were added either to induction (1 μM β-estradiol) or control solution at a concentration of 10⁸ cfu/mL. Roots from 30 plants were cut after 0-, 3- or 12-hour treatment, rinsed with sterile water, dried on a paper towel, and frozen in liquid nitrogen before RNA extraction.

Inducible VirE2-Venus Line #4 and inducible VirE2-Venus-NLS Line #4 T2 generation plants were also grown, treated, and harvested in the same manner as the inducible VirE2 plants before isolating RNA for quantitative RT-PCR (RT-qPCR) analysis.

Preparation of Samples for RNA-seq Analysis and Quantitative RT-PCR. For both RNA-seq and RT-qPCR analyses, RNA was isolated from non-induced and induced roots in the presence of Agrobacterium after 0, 3, and 12 hours of treatment using TriZoI reagent (Thermo Fisher Scientific). Three biological replicates of inducible VirE2 A. thaliana transgenic line #10 were analyzed by both RNAseq and RT-qPCR. The inducible VIP1 A. thaliana transgenic line #12 was analyzed by RNAseq and two biological replicates were analyzed by RT-qPCR (Lapham et al., 2018). Two biological replicates of inducible VirE2 Venus transgenic line #4 and inducible VirE2-Venus-NLS transgenic line #4 were analyzed by RT-qPCR. cDNA was made from polyA⁺ RNA using an Illumina TruSeq Stranded mRNA kit without rRNA depletion. One biological replicate was sequenced at the Purdue Genomics Core Facility on an Illumina HiSeq 2500 DNA sequencer using single-end, 100 cycle rapid run chemistry for the initial VirE2 pilot study and the VIP1 study. RNA from two additional VirE2 biological replicates was similarly sequenced by the Cornell University Institute of Biotechnology Genomics Facility, using an Illumina TruSeq-3′ RNA-seq kit to make cDNA.

A total of 2 μg of total RNA was treated with Ambion DNase I (Thermo Fisher Scientific) before submitting the RNA for sequencing. For RT-qPCR, cDNA was synthesized from 1.45 μg of total RNA treated with Ambion DNase I using SuperScriptIII reverse transcriptase (Thermo Fisher Scientific) following the manufacturer's protocols. RT-qPCR was performed using FastStart Essential Green Master reagents (Roche) on a Roche LightCycler 96. Primer sequences for gene amplification are listed in Supplemental Table 3. RT-qPCR data were analyzed using the LightCycler 96 software and Microsoft Excel.

RNA-seq bioinformatic analysis: Pilot Study. RNA was submitted to the Purdue Genomics Core Facility for sequencing after treatment with DNase I to remove any contaminating genomic DNA. Ribosomal RNA was depleted and cDNA libraries (stranded) were prepared from each of the samples before sequencing. Between 15 to 23 million reads were obtained for each sample (100 nucleotides per read) which were quality trimmed and mapped to the A. thaliana genome using TopHat (Trapnell et al., 2010). Differentially expressed genes were determined from the mapped (bam) files using Cuffdiff from the Cufflinks suite of programs (Trapnell et al., 2010). Custom perI scripts were used to extract genes for which fold-changes of 3 or greater occurred between the induced and non-induced control samples at their respective time points. The resulting genes were annotated by hand and separated into categories based on their Gene Ontology (GO) functions which were found in the National Center for Biotechnology Information (NCBI) database.

RNA-seq bioinformatic analysis by Purdue Bioinformatics Core: Second Study. Sequence quality was assessed using FastQC (v 0.11.7) for all samples and quality and adapter trimming was done using TrimGalore (0.4.4) (Krueger, 2017) to remove the sequencing adapter sequences and bases with Phred33 scores less than 30. The resulting reads of length 25 bases were retained (original read length=50 and lib type=unstranded) respectively. The quality trimmed reads were mapped against the reference genome using STAR (Dobin et al., 2013) (v 2.5.4b). STAR derived mapping results and annotation (GTF/GFF) file for reference genome were used as input for HTSeq (Anders et al., 2015) package (v 0.7.0) to obtain the read counts for each gene feature for each replicate. Counts from all replicates were merged using custom Perl scripts to generate a read count matrix for all samples.

The merged counts matrix was used for downstream differential gene expression analysis. Genes that did not have counts in all samples were removed from the count matrix and genes that had counts in some samples but not in others were changed from 0 to 1 in order to avoid having infinite values calculated for the fold change. Differential gene expression (DEG) analysis between treatment and control was carried out using ‘R’ (v 3.5.1) with two different methods (DESeq2 and edgeR). Basic exploration of the read count data file such as accessing data range, library sizes, etc. was performed to ensure data quality. An edgeR object was created by combining the count's matrix, library sizes, and experimental design using the edgeR (Robinson et al., 2010) (v 3.24.3) package. Normalization factors were calculated for the count's matrix, followed by estimation of common dispersion of counts. An exact test for differences between the negative binomial distribution of counts for the two experimental conditions resulted in finding differential expression, which was then adjusted for multiple hypothesis testing. DESeq2 (Love et al., 2014) (v 1.22.2) was also used to find differentially expressed genes. Both use an estimate variance-mean test based on a model using the negative binomial distribution. The significant genes were identified by examining the adjusted p-value.

Additionally, STAR mapping (bam) files were used for analysis by the Cuffdiff from Cufflinks (v 2.2.1) (Trapnell et al., 2010) suite of programs which perform DE analysis based on FPKM values. Cuffdiff uses bam files to calculate Fragments per Kilobase of exon per Million fragments mapped (FPKM) values, from which differential gene expression between the pairwise comparisons can be ascertained. Differentially expressed gene lists detected by at least two or more methods (DESeq2, edgeR, and Cufflinks) were generated using custom Perl scripts.

Gene annotations were retrieved from BioMart databases using biomartr package in ‘R’. The “transcript_biotype”, “description” attributes were extracted using mart=“plants_mart” and dataset=“athaliana_eg_gene”. GO enrichment analysis was also performed using DEGs from two or more methods while using two replicates. Singular Enrichment Analysis (SEA) from agriGO (Du et al, 2010) was used to perform GO enrichment analysis (count=5 with Fisher exact t-test with multiple testing). A GO enrichment analysis was performed using the PANTHER Classification system and online tools provided by geneontology.org.

Genotyping and Agrobacterium-mediated transient and stable transformation assays of T-DNA insertion lines. A. thaliana T-DNA insertion lines tested in this study are listed in Table 2. Seeds for these lines were obtained from the Arabidopsis Biological Resource Center (ABRC). For genotyping, DNA was isolated from leaves sampled from 10-15 individual plants after freezing the tissue in liquid nitrogen and grinding it into a fine powder using a sterile tube pestle. A total of 0.5 mL of extraction buffer (100 mM Tris pH 8.0, 50 mM EDTA, 500 mM NaCl) was added to the ground tissue before mixing thoroughly. A total of 26 μL of 20% SDS solution was added to each sample before mixing by inverting the tubes. The samples were incubated in a 65° C. water bath for 20 min and were mixed by inverting every 5 min during the incubation. After removing the samples from the water bath, 125 μL of potassium acetate buffer was added to each sample before mixing. The potassium acetate buffer is made by mixing 60 mL of 5 M KOAc from crystals, 11.5 mL glacial acetic acid, and 28.5 mL of filtered H₂O to make 100 mL (3 M of potassium and 5 M of acetate in the final solution). The tubes were placed on ice for up to 20 min before centrifugation at top speed for 10 min in a microcentrifuge at 4° C. The supernatant solution was transferred to a fresh tube (˜600 μL). The samples were centrifuged a second time if cellular debris were still evident within the supernatant solution. A 0.7 volume (420 μL) of isopropanol was added to the supernatant fluid before mixing the samples and placing them at −20° C. for at least 1 hour to precipitate the DNA. The samples were centrifuged at top speed for 10 min in a microcentrifuge at 4° C. to pellet the DNA. The DNA pellets were washed with 500 μL of 70% ethanol by flicking the tube until the pellets released from the bottom of the tube. The samples were centrifuged again for 5 min before carefully removing the ethanol. The pellets were then allowed to air-dry for 5 to 10 min before resuspending the pellets in 30 μL of 1×TE buffer (10 mM Tris-Cl, 1 mM EDTA [pH 8.0]) plus 20 μg/mL RNase A.

Lines homozygous for the annotated T-DNA insertions were confirmed by PCR (primer sequences are listed in Table 8). PCR reaction mixes were made using ExTaq Buffer (TaKaRa), dNTPs (0.2 mM), the appropriate forward and reverse primers (0.2 μM each), homemade Taq polymerase, and water with a tenth volume of sample added to act as a template. The reactions were incubated at 95° C. for 3 min before performing 35 cycles of a 30 sec, 95° C. denaturation step, followed by a 30 sec annealing step (temperature was about 5° C. lower than the average melting temperature for each primer set), and a 1 min, 72° C. extension step (1 min). A final 10 min extension step at 72° C. followed the last cycle before PCR products were visualized using gel electrophoresis.

A. thaliana plants homozygous for their annotated T-DNA insertion were grown for 20 days in baby food jars containing sterile Gamborg's B5 medium before cutting their roots into 3-5 mm segments. The segments were assayed as described in Tenea et al. (2009). A. tumefaciens At849 (GV3101::pMP90 containing pBISN1) was used to measure transient transformation, whereas A. tumefaciens A208 (Sciaky et al., 1978) was used for stable transformation (Supplemental Table 2). Three replicates were assayed for each experiment with root segments from 10 plants pooled for each replicate. A minimum of 80 root segments were scored for each data point and statistical analysis was performed using ANOVA.

Protein Isolation and Proteomics Analysis. Roots were homogenized in 8 M urea using a Percellys® 24 homogenizer (Bertin) and incubated at room temperature for 1 hour with continuous vortexing before centrifugation at 14,000 rpm for 15 min at 4° C. The supernatant solution was transferred to a new tube and the protein concentration was determined using a Pierce™ BCA assay (Thermo Fisher Scientific). A total of 100 μg protein from each sample (equivalent volume) was taken for digestion. Proteins were first precipitated using 4 volumes of cold acetone (−20° C.) overnight before centrifugation at 14,000 rpm for 15 min at 4° C. to collect the precipitated proteins. Protein pellets were washed twice with 80% cold (−20° C.) acetone, dried in a speed-vac for 5 min, and then solubilized in 8 M urea. Samples were reduced using 10 mM dithiotreitol and cysteine alkylated using 20 mM iodoacetamide. This was followed by digestion using sequence grade Lyc-C/Trypsin (Promega) mix at a 1:25 (enzyme:substrate) ratio to enzymatically digest the proteins. All digestions were carried out at 37° C. overnight. The samples were cleaned over C18 MicroSpin columns (Nest Group), dried, and resuspended in 97% purified H₂O/3% acetonitrile (ACN)/0.1% formic acid (FA). After BCA at the peptide level, 1 μg of each sample was loaded onto the column.

Digested samples were analyzed using a Dionex UltiMate 3000 RSLC Nano System coupled with a Q Exactive™ HF Hybrid Quadrupole-Orbitrap Mass Spectrometer (Thermo Scientific, Waltham, Mass.). Peptides were first loaded onto a 300 μm×5 mm C18 PepMap™ 100 trap column and washed with 98% purified water/2% acetonitrile (ACN)/0.01% formic acid (FA) using a flow rate of 5 μL/min. After 5 min, the trap column was switched in-line with a 75 μm×50 cm reverse phase Acclaim™ PepMap™ RSLC C18 analytical column heated to 50° C. Peptides were separated over the analytical column using a 120 min method at a flow rate of 300 nL min⁻¹. Mobile phase A contained 0.01% FA in purified water while mobile phase B consisted of 0.01% FA/80% ACN in purified water. The linear gradient began at 2% B and reached 10% B in 5 min, 30% B in 80 min, 45% B in 91 min, and 100% B in 93 min. The column was held at 100% B for the next 5 min before returning to 5% B where it was equilibrated for 20 min. Samples were injected into the QE HF through the Nanospray FIex™ Ion Source fitted with an emitter tip from New Objective. MS spectra were collected from 400 to 1600 m/z at 120,000 resolution, a maximum injection time of 100 ms, and a dynamic exclusion of 15 s. The top 20 precursors were fragmented using higher-energy C-trap dissociation (HCD) at a normalized collision energy of 27%. MS/MS spectra were acquired in the Orbitrap at a resolution of 15,000 with a maximum injection time of 20 ms. The raw data were analyzed using MaxQuant software (v. 1.5.3.28) against a TAIR 10 protein database combined with VirE2 proteins (Cox et al., 2008; 2011; 2014). The search was performed with the precursor mass tolerance set to 10 ppm and MS/MS fragment ions tolerance was set to 20 ppm. The enzyme was set to trypsin and LysC, allowing up to two missed cleavages. Oxidation of methionine was defined as a variable modification, and carbamidomethylation of cysteine was defined as a fixed modification. The “unique plus razor peptides” (razor peptides are the non-unique peptides assigned to the protein group with the most other peptides) were used for peptide quantitation. The false discovery rate (FDR) of peptides and proteins identification was set at 0.01. iBAQ scores and MS/MS counts for each identified protein were compared between the non-induced and induced samples. Proteins which showed a 0.2-fold (20%) increase or decrease in abundance in the induced versus non-induced samples for at least two biological replicates by comparing both iBAQ scores and MS/MS counts were considered to have levels which changed in response to VirE2 induction.

RNAseq data deposition. RNAseq data have been deposited at the NCBI GEO with the accession number GSE172314.

### Example 2

### Cytoplasmic but not Nuclear Localized VirE2 can Support Transformation

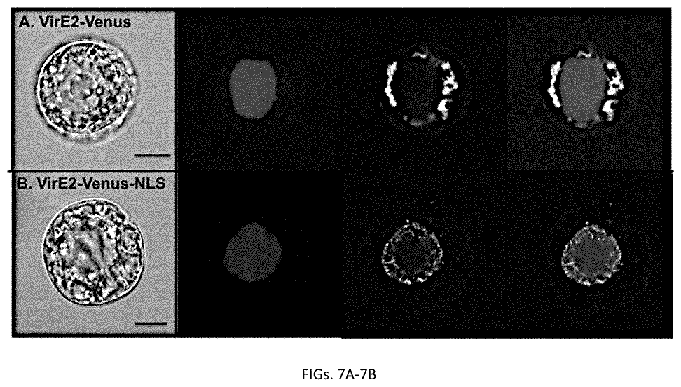

To determine the subcellular localization of VirE2 that is required to facilitate transformation, plasmids were first constructed to express the recombinant proteins VirE2-Venus or VirE2-Venus-NLS (containing a nuclear localization signal [NLS]) constitutively. Tobacco BY-2 protoplasts were individually co-transfected with DNA from each of these constructs and a plasmid containing a red fluorescence protein (RFP) nuclear marker. The protoplasts were imaged 16 hours later using confocal microscopy (FIGS. 7A-7B). Consistent with data from previous studies (Bhattacharjee et al., 2008; Grange et al., 2008; Lee et al., 2008; Li et al., 2014, 2020; Shi et al., 2014; Li and Pan, 2017; Lapham et al., 2018; Roushan et al., 2018), VirE2-Venus localized to the cytoplasm (FIG. 7A); however, VirE2-Venus-NLS localized to the nucleus (FIG. 7B). Although where in the cytoplasm VirE2-Venus localizes, it does not localize to the nucleus, and for convenience, the subcellular localization of VirE2-Venus is referred as “cytoplasmic”.

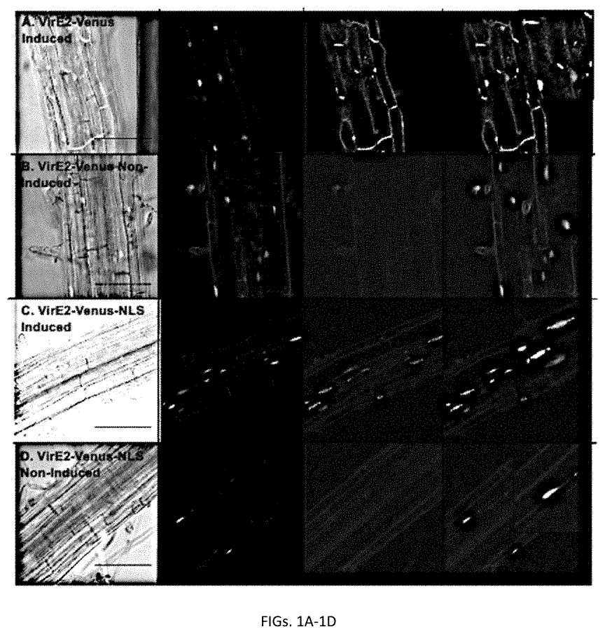

Transgenic A. thaliana plant lines were generated that express either VirE2-Venus or VirE2-Venus-NLS under the control of a p-estradiol inducible promoter (Zuo et al., 2000), and a Cerulean-NLS nuclear marker under the control of a constitutive Cauliflower Mosaic Virus (CaMV) double 35S promoter. After incubating the plants in either control (non-induced) or p-estradiol (induced) solution for 9 hours, the roots were imaged using confocal microscopy. Only induced, but not non-induced, roots showed a fluorescence signal (FIG. 1A & FIG. 1C), whereas the Cerulean marked nuclei were evident in both non-induced and induced roots. VirE2-Venus localized outside of the nucleus and throughout the cytoplasm (FIG. 1A), whereas VirE2-Venus-NLS co-localized with the Cerulean nuclear marker (FIG. 1C) in transgenic Arabidopsis roots.

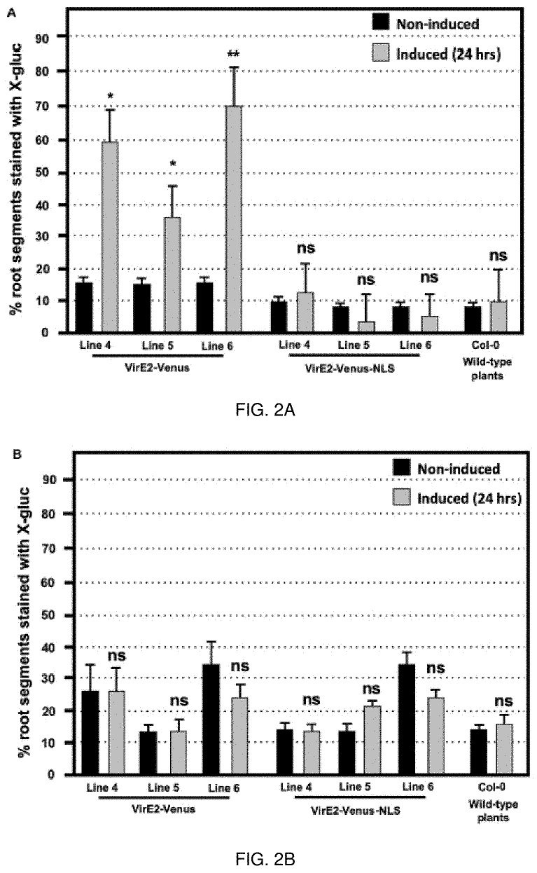

Transient Agrobacterium-mediated transformation assays were performed on wild-type (Col-0), and three independent lines each of inducible VirE2-Venus, and inducible VirE2-Venus-NLS transgenic plants. These lines were chosen based upon equivalent levels of expression of the fluorescently-tagged VirE2 protein. Plant roots were treated with either control or β-estradiol solution for 24 hours before cutting the roots into small segments and infecting them with a virE2 mutant Agrobacterium strain containing the T-DNA binary vector pBISN2 or a virE2⁺ control strain containing pBISN1. The T-DNAs of pBISN1 and pBISN2 are identical and contain a plant-active gusA-intron gene (Narasimhulu et al., 1996). A low level of transformation was observed in all non-induced samples infected with the virE2 mutant Agrobacterium strain (FIG. 2A). Such low-level virE2-independent transformation has been observed previously (Stachel and Nester, 1986; Rossi et al., 1996; Dombek and Ream, 1997). Induction of only transgenic plants encoding cytoplasmic-localized VirE2-Venus, but not nuclear-localized VirE2-Venus-NLS, increased transient transformation efficiency compared to that of non-induced levels. The inability of nuclear-localized VirE2-Venus-NLS to complement the virE2 mutant strain to full virulence was not due to a toxic effect of the protein because both inducible VirE2-Venus and inducible VirE2-Venus-NLS plants showed comparable transformation rates when infected with a virE2⁺ Agrobacterium strain (FIG. 2B). In addition, the similar transformation efficiencies of induced VirE2-Venus and VirE2-Venus-NLS plants by a virE2⁺ Agrobacterium strain indicates that nuclear-localized VirE2 does not prevent T-strand uncoating in the nucleus, thus preventing gusA transgene expression. Thus, these results indicate that in order for VirE2 to complement the transformation deficiency of a virE2 mutant Agrobacterium strain, it must be localized in the cytoplasm.

### Example 3

### Cytoplasmic-Localized VirE2 Alters Expression of Numerous Arabidopsis Genes Involved in Defense Response and Transformation Susceptibility

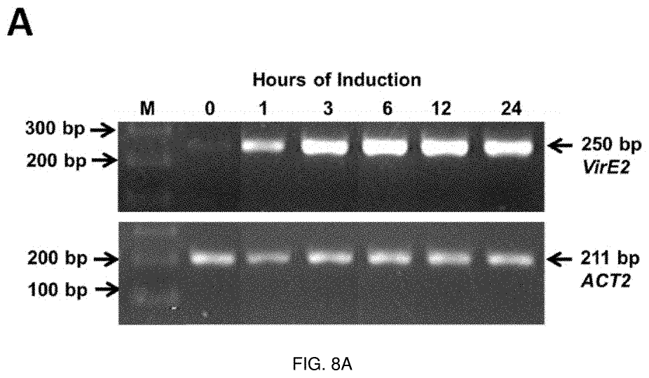

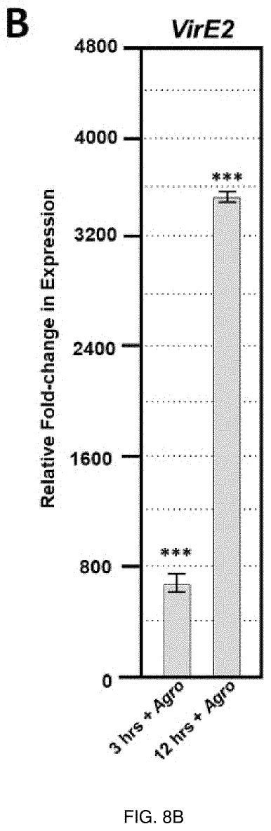

Multiple transgenic A. thaliana lines were generated that express untagged VirE2 under the control of a β-estradiol inducible promoter and VirE2 induction was tested by RT-PCR. Root tissue pooled from about 30 plants of one representative line was harvested after treating either with β-estradiol or control (non-induced) solution for 3 or 12 hr. Both the control and β-estradiol solutions contained the avirulent strain A. tumefaciens A136 that lacks a Ti-plasmid (Sciaky et al., 1978) at a concentration of 10⁸ cfu/mL. The inclusion of this bacterial strain was done to mimic more closely natural infection conditions because a plant cell is only exposed to VirE2 in the presence of Agrobacterium. RNA was extracted from each sample and induced expression of VirE2 was confirmed using RT-PCR. VirE2 transcripts were detectable within 1 hour of induction (FIG. 8A). RT-qPCR was performed on samples collected from 3 and 12 hours after induction (FIG. 8B) before submitting the samples for RNA-seq analysis. This analysis was initially performed on one biological replicate composed of roots from about 30 plants as a pilot study to identify potential target genes to test for transformation phenotypes. Differentially expressed genes were determined using Cufflinks (Trapnell et al., 2012). For this pilot study and considering all time points, a total of 443 A. thaliana genes (about 1.5% of the annotated protein coding genome) were differentially expressed in VirE2-induced versus non-induced samples.

RNAseq analysis was conducted on two additional biological replicates, each composed of roots from about 30 plants of the same inducible VirE2 line. Using more stringent criteria than used in our pilot study, total 145 unique up-regulated genes and 25 unique down-regulated genes were identified in induced versus non-induced samples by at least two computational methods with an adjusted P-value cut-off of 0.1 across all analyses. Of these unique 170 differentially expressed genes (DEGs), 61 were identified in the pilot study (Table 1). DEGs identified in both studies were displayed according to their annotated Gene Ontology (GO) biological process (FIGS. 3A-3B; Ashburner et al., 2000). Some genes which showed significant changes in expression were tested using RT-qPCR to validate the RNA-seq results. All genes tested by RT-qPCR showed changes in expression consistent with the RNA-seq data, FIGS. 9A-9H, Table 6).

TABLE 6
VirE2 differentially expressed genes tested using RT-qPCR

| Gene Name | Gene_ID | Encoded Protein |
|---|---|---|
| ADH1 | At1g77120 | Alcohol dehydrogenase 1 |
| PRKP | At1g51840 | Protein kinase-related protein |
| IncRNA | At3g25795 | Trans-acting siRNA 4 |
| PR | At4g33720 | Putative pathogenesis-related protein |
| LSU1 | At3g49580 | Response to low sulfur 1 |
| LRRPK | At1g51830 | Putative leucine-rich repeat protein kinase |
| AGP21 | At1g55330 | Arabinogalactan protein 21 |
| NTR2.6 | At3g45060 | High affinity nitrate transporter 2.6 |

Genes involved in response to stress (both biotic and abiotic), regulation of gene expression, biological regulation, and various other developmental, biosynthetic, and metabolic processes were differentially expressed in VirE2-induced plants (FIG. 3A). A subset of genes differentially expressed following VirE2 induction are involved in defense responses, particularly those involved in responding to bacteria (FIG. 3B).

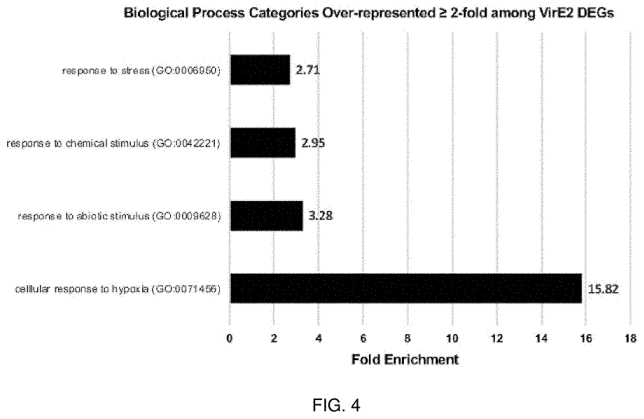

A GO enrichment analysis was performed to determine which categories of genes were over-represented 2-fold or more in the RNA-seq dataset for those 61 DEGs common to both RNAseq experiments (FIG. 4; Table 1). Genes involved in cellular response to hypoxia, abiotic and chemical stimuli, and stress were enriched. Some stress associated genes, such as those encoding protein phosphatase 2C (downregulated) and HEAT SHOCK PROTEIN 90.1 (upregulated), have previously been shown to be important for transformation (Tao et al., 2004; Park et al. 2014). These VirE2-induced changes may facilitate transformation.

It is possible that the differential expression of Arabidopsis genes following VirE2 induction is merely a stress response to overexpression of a protein in the plant cytoplasm, as indicated by induction of the HSP90.1 gene. To control for this possibility, Arabidopsis lines were generated that inducibly overexpress VIP1 (Lapham et al., 2018). VIP1 encodes a protein that localizes both to the nucleus and the cytoplasm, depending upon the time after osmotic or thigomostimulation (Tsugama et al., 2012, 2014, 2016). Unlike VirE2, VIP1 is not important for Agrobacterium-mediated transformation (Shi et al., 2014; Lapham et al., 2018). RNAseq analysis of RNA from Arabidopsis roots extracted was conducted at various times after VIP1 induction and VirE2-differentially expressed genes with VIP1-differentially expressed genes were compared. Under the same conditions (in the presence of Agrobacterium, and at the same time point), only two DEG (At3G13437 and At4G26200) overlap between the VirE2 and VIP1 overexpression analyses, and neither of these encode stress-response or heat shock/chaperonin proteins. Thus, overexpression of VirE2 elicits a specific DEG response that differs from that elicited by overexpression of another protein.

TABLE 1
VirE2 differentially expressed genes in both RNAseq studies

| | | Up/Down-regulated | Up/Down-regulated |
|---|---|---|---|
| | | Second study | Pilot study |
| Gene ID | Encoded Protein | (Fold-change) | (Fold-change) |
| | VirE2 | Up (194.0)-3 hours | Up (188)-3 hours |
| | | Up (2342.3)-12 hours | Up (Up 1961.7)-12 hours |
| At1g01580 | Ferric reduction oxidase 2 | Down (2.5)-3 hours | Down (1.4)-3 hours |
| | | Down (2.4)-12 hours | Down (2.5)-12 hours |
| At1g09932 | Phosphoglycerate mutase | Up (1.8) | Up (3.6) |
| | family protein | | |
| At1g14200 | RING/U-box superfamily | Up (3.4) | Up (1.9) |
| | protein SNIPER1 | | |
| At1g23730 | Beta carbonic anhydrase 3 | Up (5.2) | Up (2.8) |
| At1g26800 | E3 ubiquitin-protein ligase | Up (3.0) | Up (1.4) |
| | MPSR1 | | |
| At1g32350 | Alternative oxidase 1D | Up (6.9) | Up (2.5) |
| At1g61560 | MILDEW RESISTANCE | Up (1.9) | Up (1.7) |
| | LOCUS O 6 | | |
| At1g61820 | Beta-glucosidase 46 | Up (1.9) | Up (1.8) |
| At1g62370 | RING/U-box superfamily | Up (2.5) | Up (1.9) |
| | protein | | |
| At1g63530 | Hypothetical protein | Up (3.0) | Up (1.5) |
| At1g66090 | Disease resistance protein | Up (4.6) | Up (6.3) |
| | (TIR-NBS class) | | |
| At1g73120 | F-box/RN I superfamily protein | Down (5.5) | Down (5.7) |
| At2g16660 | Major facilitator superfamily | Down (2.4) | Down (3.8) |
| | protein | | |
| At2g17040 | NAC domain containing | Up (2.6) | Up (5.0) |
| | protein 36 | | |
| At2g23270 | Transmembrane protein | Up (2.9) | Up (4.0) |
| At2g26150 | Heat stress transcription | Up (11.6) | Up (1.7) |
| | factor A-2 | | |
| At2g28160 | Transcription factor FER-LIKE | Up (1.8) | Up (1.4) |
| | IRON DEFICIENCY-INDUCED | | |
| | TRANSCRIPTION | | |
| | FACTOR | | |
| At2g29450 | Glutathione 5-transferase tau 5 | Up (2.4) | Up (2.7) |
| At2g42850 | Cytochrome P450, family 718 | Up (2.1) | Up (1.4) |
| At2g44010 | Hypothetical protein | Up (1.6) | Up (3.2) |
| At2g44578 | RING/U-box superfamily | Up (2.9) | Up (1.6) |
| | protein | | |
| At2g45920 | U-box domain-containing | Up (1.9) | Up (1.4) |
| | protein | | |
| At3g07090 | PPPDE putative thiol | Up (2.0) | Up (1.4) |
| | peptidase family protein | | |
| At3g09290 | Telomerase activator1 | Up (3.2) | Up (2.2) |
| | (TAC1) | | |
| At3g09350 | Fes1A | Up (3.2) | Up (1.3) |
| At3g13437 | Enhancer of vascular Wilt | Up (2.2) | Up (3.6) |
| | Resistance 1; EWR1 | | |
| At3g14362 | DEVIL 19; DVL19; | Up (2.6) | Up (1.7) |
| | ROTUNDIFOLIA like 10 | | |
| At3g15340 | Proton pump interactor 2 | Up (2.8) | Up (1.3) |
| | (PPI2) | | |
| At3g29000 | Calcium-binding EF-hand | Up (2.8) | Up (2.6) |
| | family protein | | |
| At3g48920 | Myb domain protein 45 | Up (2.1) | Up (3.4) |
| At3g46810 | Cysteine/Histidine-rich C1 | Down (2.7) | Down (2.7) |
| | domain family protein | | |
| At3g53150 | UDP-glucosyl transferase | Up (2.3) | Up (1.5) |
| | 73D1 | | |
| At3g54150 | Embryonic abundant protein- | Up (2.4) | Up (1.8) |
| | like | | |
| At3g61400 | 1-aminocyclopropane-1-carboxylate | Down (9.7) | Down (2.7) |
| | oxidase homolog 8 | | |
| At4g04990 | Serine/arginine repetitive | Up (2.1) | Up (2.4) |
| | matrix-like protein (DUF761) | | |
| At4g19690 | Fe(2+) transport protein 1 | Down (2.5)-3 hours | Down (2.3)-3 hours |
| | | Down (2.8)-12 hours | |
| At4g26200 | 1-aminocyclopropane-1- | Up (4.8) | Up (1.7) |
| | carboxylate synthase 7 | | |
| At4g30230 | Uncharacterized protein | Up (26.2) | Up (2.1) |
| | At4g30230 | | |
| At4g30960 | CBL-interacting | Up (1.6) | Up (1.4) |
| | serine/threonine-protein | | |
| | kinase 6 | | |
| At4g33050 | Calmodulin-binding family | Up (1.9) | Up (1.3) |
| | protein | | |
| At4g34950 | Major facilitator superfamily | Down (2.3) | Down (3.7) |
| | protein | | |
| At4g37290 | Transmembrane protein | Up (5.1) | Up (5.7) |
| At4g39670 | ACD11 homolog protein | Up (2.1) | Up (1.5) |
| At5g02490 | Probable mediator of RNA | Up (2.3) | Up (2.8) |
| | polymerase II transcription | | |
| | subunit 37c | | |
| At5g03545 | Expressed in response to | Down (1.6) | Down (2.3) |
| | phosphate starvation protein | | |
| At5g06760 | LEA4-5 | Up (8.3)-3 hours | Up (6.6)-12 hours |
| | | Up (4.4)-12 hours | |
| At5g13320 | Auxin-responsive GH3 family | Up (8.3) | Up (27.3) |
| | protein | | |
| At5g25450 | Cytochrome b-c1 complex | Up (2.7) | Up (3.3) |
| | subunit 7 | | |
| At5g39050 | Phenolic glucoside | Up (2.4) | Up (1.4) |
| | malonyltransferase 1 | | |
| At5g39360 | EID1-like 2 | Up (1.7) | Up (1.5) |
| At5g39670 | Probable calcium-binding | Up (2.5) | Up (1.9) |
| | protein CML46 | | |
| At5g40010 | AAA-ATPase ASD, | Up (2.6) | Up (1.3) |
| | mitochondrial | | |
| At5g43450 | 1-aminocyclopropane-1- | Up (3.9) | Up (3.4) |
| | carboxylate oxidase homolog 10 | | |
| At5g45840 | Phytosulfokin receptor 1 | Up (2.5) | Up (1.3) |
| At5g51440 | 23.5 kDa heat shock protein, | Up (5.9) | Up (2.1) |
| | mitochondrial | | |
| At5g54165 | Avr9/Cf-9 rapidly elicited | Up (4.7) | Up (21.4) |
| | protein | | |
| At5g57010 | IQ domain-containing protein | Up (5.4) | Up (1.6) |
| | IQM5 | | |
| At5g57510 | Cotton fiber protein | Up (4.4) | Up (2.4) |
| At5g59820 | Zinc finger protein ZAT12 | Up (2.0) | Up (1.3) |
| At5g64810 | Probable WRKY transcription | Up (3.0) | Up (1.7) |
| | factor 51 | | |

### Example 4

### Arabidopsis Lines Harboring Mutations in Some Genes Differentially Expressed by VirE2 Exhibit Altered Transformation Phenotypes

T-DNA insertion mutant lines of a subset of the VirE2 differentially expressed genes identified in our pilot RNA-seq study were tested for transformation susceptibility (Table 2). Transformation results for mutants of VirE2 upregulated and downregulated genes are shown in FIGS. 10A-10H and FIGS. 11A-11F, respectively, and are summarized in Table 2. If a mutant showed no statistically significant difference in transformation efficiency at any of the tested bacterial concentrations, the results are reported as “No change” in Table 2. However, some of these mutations may still have a minor impact on transformation.

The atpsk3, tst18, and miR163 mutant lines (Table 2; FIGS. 10B, 10C, 10G) showed decreased transformation compared to that of wild-type plants. All three genes are upregulated in the presence of VirE2 and may therefore facilitate transformation. The pr5 mutant showed an increase in transient transformation (Table 2; FIG. 10D). PR5 is up-regulated in the presence of VirE2, and because of its role in defense response and effector-triggered immunity (ETI; Wu et al., 2014) one would predict that the pr5 mutant would be more susceptible to Agrobacterium-mediated infection. At least for transient transformation, this prediction is consistent with our results (FIG. 10D).

Several of the mutants for genes down-regulated in the presence of VirE2 showed increased transient or stable transformation efficiency compared to that of wild-type plants (FIGS. 11B-11F). These genes may act to inhibit transformation, and their VirE2-dependent down-regulation may facilitate transformation, as reflected by the increased susceptibility of their respective knockout mutant lines to Agrobacterium-mediated transformation. A Protein phosphatase 2C (FIG. 11F) was previously identified as a transformation inhibitor (Tao et al., 2004). Conversely, the exl1, oep6, and rld17 mutants showed decreased transformation (Table 2; FIGS. 11A, 11D, 11E) even though they are downregulated in the presence of VirE2. These genes are important for transformation, but their mechanism of action and regulation during transformation remain unknown.

TABLE 2
Transformation phenotypes of mutants of VirE2 differentially expressed genes

| | | | Up/Down- | | |
|---|---|---|---|---|---|
| | | | regulated | | |
| Gene | | | (Fold- | | Transformation |
| Name | Gene_ID | Encoded Protein | change) | ABRC Stock ID | Result |
| IncRNA | At3g12965 | Long non-coding | Up (5.8) | SALK_086573 | No change |
| | | RNA | | | |
| atpsk3 | At3g44735 | Phytosulfokine 3 | Up (5) | SALK_044781 | *Decreased |
| | | precursor | | | transient |
| acs6 | At4g11280 | 1-aminocyclopropane- | Up (3) | SALK_054467 | No change |
| | | 1-carboxylate | | | |
| | | synthase 6 | | | |
| tst18 | At5g66170 | Thiosulfate | Up (3.7) | CS867285 | *Decreased |
| | | sulfurtransferase 18 | | | transient and |
| | | | | | stable |
| pr5 | At1g75040 | Pathogenesis- | Up (14) | SALK_055063C | *Increased |
| | | related protein 5 | | | transient |
| agp14 | At5g56540 | Arabinogalactan | Up (4.9) | SALK_096806 | No change |
| | | protein 14 | | | |
| tasi4 | At3g25795 | Trans-acting siRNA 4 | Up (15.1) | SALK_066997 | No change |
| miR163 | At1g66725 | microRNA 163 | Up (3.3) | CS879797 | **Decreased |
| | | | | | stable |
| samp | At2g41380 | S-adenosyl-L- | Up (10.1) | SALK_209995C | No change |
| | | methionine- | | | |
| | | dependent | | | |
| | | methyltransferases | | | |
| | | superfamily protein | | | |
| tasi3 | At3g17185 | Trans-acting siRNA 3 | Up (3) | GABI-Kat Stock | No change |
| | | | | N432182 | |
| | | | | (N2051875) | |
| ex11 | At1g23720 | Proline-rich | Down (3.3) | SALK_010243C | **Decreased |
| | | extensin-like family | | | stable |
| | | protein 1 | | | |
| mee39 | At3g46330 | Maternal effect | Down (4.7) | SALK_065070C | No change |
| | | embryo arrest 39 | | | |
| | | (putative LRR | | | |
| | | receptor-like | | | |
| | | serine/threonine- | | | |
| | | protein kinase) | | | |
| rbc3b | At5g38410 | Ribulose | Down (7.4) | SALK_117835 | No change |
| | | bisphosphate | | | |
| | | carboxylase small | | | |
| | | chain 3B | | | |
| abah3 | At5g45340 | Abscisic acid 8′- | Down (3.4) | SALK_078170 | *Increased |
| | | hydroxylase 3 | | | transient |
| ntr2.6 | At3g45060 | High affinity nitrate | Down (28) | SALK_2041010 | *Increased |
| | | transporter 2.6 | | | transient |
| cup | At3g60270 | Cupredoxin | Down (31.3) | SALK_2014440 | **Increased |
| | | superfamily protein | | | transient |
| ntr2:1 | At1g08090 | Nitrate transporter | Down (35.7) | SALK_035429C | *Increased |
| | | 2:1 | | | transient |
| oep6 | At3g63160 | Outer envelope | Down (5.6) | CS862774 | *Decreased |
| | | protein 6 | | | stable |
| | | (chloroplast) | | | |
| esm1 | At3g14210 | Epithiospecifier | Down (10) | SALK_150833C | **Increased |
| | | modifier 1 | | | stable |
| rld17 | At2g17850 | Rhodanese-like | Down (4.7) | SALK_115776C | ***Decreased |
| | | domain-containing | | | transient and |
| | | protein 17 | | | stable |
| pp2c25 | At2g30020 | Putative protein | Down (3.5) | SALK_104445 | **Increased |
| | | phosphatase 2C 25 | | | transient |
| adh1 | At1g77120 | Alcohol | Down (23.2) | SALK_052699 | ***Increased |
| | | dehydrogenase 1 | | | transient |

ANOVA test *Pvalue < 0.05, **Pvalue < 0.01, ***Pvalue < 0.001.

### Example 5

### The Subcellular Location of VirE2 Results in Different Arabidopsis Root Transcriptome Patterns

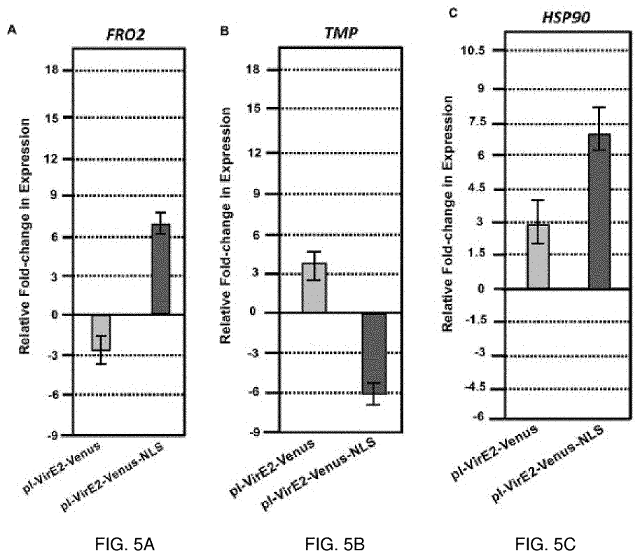

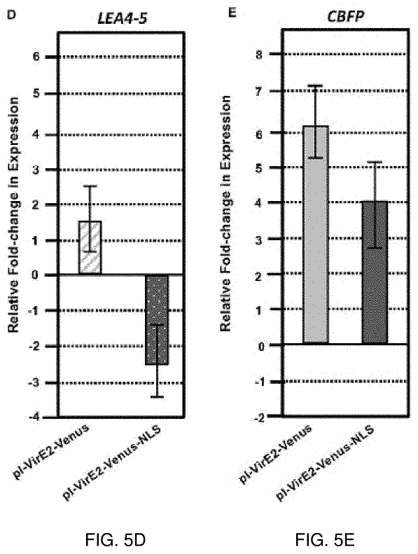

Roots of transgenic inducible VirE2-Venus or VirE2-Venus-NLS plants were induced for 3 or 12 hours with β-estradiol in the presence of A. tumefaciens A136. Total RNA was extracted from infected root samples. A subset of genes which exhibited significant changes in expression after the induction of an untagged VirE2 line (determined by RNAseq) was tested by RT-qPCR for changes in expression in the inducible VirE2-Venus and inducible VirE2-Venus-NLS samples compared with non-induced samples (Table 3, FIGS. 5A-5E). The inducible VirE2-Venus (cytoplasmic localized) samples showed either no change or a similar pattern of gene expression changes to those observed for untagged VirE2 (Table 1, FIGS. 5A-5E). However, the genes FERRIC REDUCTION OXIDASE2 (FRO2), TRANSMEMBRANE PROTEIN (TMP), and LATE EMBRYOGENESIS ABUNDANT 4-5 (LEA4-5) showed the opposite pattern of gene expression changes in the VirE2-Venus-NLS (nuclear localized) line compared to that of the VirE2-Venus (cytoplasmic localized) samples (FIGS. 5A, 5B, and 5D). These results suggest that the changes in expression of these genes resulted from the cytoplasmic localization of VirE2. Interestingly, both cytoplasmic (VirE2-Venus) and nuclear-localized (VirE2-Venus-NLS) VirE2 caused up-regulation of HEAT SHOCK PROTEIN 90-1 (HSP90) and CALMODULIN-BINDING FAMILY PROTEIN (CBFP; FIGS. 5C and 5E), but to different extents. Up-regulation of these genes occurred after 12 hours of induction and could have resulted from downstream effects caused by the presence of VirE2 in the plant regardless of localization. It is also possible that a small amount of VirE2 or VirE2-Venus could have entered the nucleus and was sufficient to induce expression of these two genes.

TABLE 3
VirE2 subcellular localization impacts changes in plant gene expression

| | | | | | Up/Down- |
|---|---|---|---|---|---|
| | | | | | regulated |
| | | | | Up/Down- | in the |
| | | | Up/Down- | regulated in | presence of |
| | | | Regulated in the | the presence | VirE2- |
| Gene | | | presence of VirE2 | of VirE2-Venus | Venus-NLS |
| Name | Gene_ID | Encoded Protein | (untagged) | (cytoplasmic) | (nuclear) |
| FRO2 | At1g01580 | FERRIC | Down 2-fold | Down 2.9-fold | Up 6.3-fold |
| | | REDUCTION | | | |
| | | OXIDASE 2 | | | |
| TMP | At4g37290 | TRANSMEMBRANE | Up 5-fold | Up 3.9-fold | Down 6.9-fold |
| | | PROTEIN | | | |
| HSP90 | At5g52640 | HEAT SHOCK | Up 6-fold | Up 2.9-fold | Up 7.1-fold |
| | | PROTEIN 90-1 | | | |
| LEA4-5 | At5g06760 | LATE | Up 3-fold | Up 1.6-fold | Down 2.5-fold |
| | | EMBRYOGENESIS | | | |
| | | ABUNDANT 4-5 | | | |
| CBFP | At5g57010 | CALMODULIN- | Up 5-fold | Up 6.1-fold | Up 4.0-fold |
| | | BINDING FAMILY | | | |
| | | PROTEIN | | | |

### Example 6

### VirE2 Alters the Arabidopsis Proteome to Facilitate Transformation

The effect of VirE2 on the Arabidopsis root proteome was identified using the same transgenic inducible VirE2 Arabidopsis line for which transcriptome analysis was employed. A total of 135 unique A. thaliana proteins (about 0.6% of the detectable proteins) showed a statistically significant change in abundance of at least 20% in all three biological replicates of VirE2-induced samples. These proteins were graphed according to their annotated Gene Ontology (GO) biological process (FIGS. 6A-6D; Ashburner et al., 2000). Proteins previously shown to be important for transformation, such as histones and histone modifying proteins, arabinogalactan proteins, and cyclophilins, showed increased abundance in the presence of VirE2 (Table 4; Deng et al., 1998; Nam et al., 1999; Gaspar et al., 2004; Crane and Gelvin, 2007; Tenea et al., 2009). These VirE2-induced elevations in protein level likely facilitate transformation. Proteins whose levels changed in the presence of VirE2 did not show changes in their RNA levels, suggesting that VirE2-induced changes to protein levels occur at the translational or post-translational level.

TABLE 4
Proteins previously identified as important for transformation
show increased abundance in the presence of VirE2

| | | | % Change in |
|---|---|---|---|
| | | | Protein Level |
| | Gene | | (Time Post-VirE2 |
| Gene ID | Name | Encoded Protein | Induction)^{a} |
| At2g28740 | HIS4 | Histone H4 | +37% (3 hours); |
| | (formerly | | p = 0.006 |
| | HFO4) | | |
| At4g27230 | HTA2 | Histone H2A2 | +140% (3 hours); |
| | | | p = ns |
| At5g03740 | HD2C | Histone | +35% (3 hours) |
| | (formerly | deacetylase 2C | p = 0.007 |
| | HDT3) | | |
| At3g44750 | HDA3 | Histone | +50% (12 hours) |
| | (formerly | deacetylase 3 | p = 0.002 |
| | HDT1) | | |
| At2g16600 | ROC3 | Rotamase | +20% (12 hours) |
| | | cyclophilin 3 | p = 0.061 |
| At3g56070 | ROC2 | Rotamase | +85% (12 hours) |
| | | cyclophilin 2 | p = 0.046 |
| At1g03870 | FLA9 | FASCICLIN-like | +25% (3 hours) |
| | | arabinogalactan 9 | p = 0.035 |
| At1g28290 | AGP31 | Arabinogalactan | +50% (3 hours) |
| | | protein 31 | p = ns |

^{a}p-value according to iBAQ analysis; ns, not significant at p < 0.1.

Transgenic lines of A. thaliana were generated that constitutively overexpressed selected genes whose proteins showed increased abundance in response to VirE2-induction (Table 5). Although statistical analysis of the iBAC (intensity-Based Absolute Quantitation) scores showed that the increased ARABINOGALACTAN PROTEIN 31 (AGP31) abundance was statistically significant at only p=0.27, this gene was included in the overexpression analysis because the previous study indicated that arabinogalactan proteins were important for transformation (Gaspar et al., 2004). Roots from multiple T2 generation transgenic lines were assayed for transient and stable transformation susceptibility (Table 5; FIGS. 12A-12E). Some transgenic lines containing a PEROXIDASE 34 (PERX34) overexpressing construct showed decreased stable transformation (Table 5; FIG. 12A), whereas most of the ROTAMASE CYCLOPHILIN 2 (ROC2) overexpression lines showed increased transient transformation (Table 5; FIG. 12B). Transgenic lines containing an overexpression construct for HISTONE DEACTYLASE 3 (HDA3: FIG. 12C) showed increased transient transformation. Some plant lines containing overexpression constructs for both HISTONE DEACTYLASE 2C (HD2C: FIG. 12D) and AGP31 (FIG. 12E) also showed increased transformation efficiency. These data suggest that VirE2-induced changes to levels of specific proteins may facilitate transformation.

TABLE 5
Transformation phenotypes of A. thaliana lines containing overexpression constructs
of genes whose proteins show increased abundance post-VirE2 induction

| | | | % Change in | |
|---|---|---|---|---|
| | | | Protein Level | |
| | Gene | Encoded | (Time Post-VirE2 | Transformation |
| Gene ID | Name | Protein | Induction) | Result |
| At3g49120 | PERX34 | Peroxidase 34 | +38% (3 hours) | *Decreased stable |
| At5g03740 | HD2C | Histone | +35% (3 hours) | **Increased |
| | (formerly | deacetylase 2C | | transient and |
| | HDT3) | | | *Increased stable |
| At3g44750 | HDA3 | Histone | +50% (12 hours) | *Increased |
| | (formerly | deacetylase 3 | | transient |
| | HDT1) | | | |
| At3g56070 | ROC2 | Rotamase | +85% (12 hours) | **Increased |
| | | cyclophilin 2 | | transient |
| At1g28290 | AGP31 | Arabinogalactan | +50% (3 hours) | *Increased |
| | | protein 31 | | transient |

ANOVA test *Pvalue < 0.05, **Pvalue < 0.01.

Taken together, these results suggest that VirE2 impacts the plant cell on both the RNA and protein levels to facilitate transformation, and that the effect of VirE2 occurs from its position in the plant cytoplasm.

VirE2 alters the steady-state levels of specific plant RNAs and proteins which are known to be important for transformation. VirE2 mediates these changes post-transcriptionally. This model is supported by the rapid changes in levels of certain proteins and more delayed changes in levels of specific RNAs observed in response to VirE2 induction. Coupled with the observation that cytoplasmic localization of VirE2 is required for it to function in transformation, these results are consistent with a post-transcriptional role in modulating mRNA and protein levels. It is now concluded that VirE2, from its location in the plant cytoplasm, modulates specific plant steady-state RNA and protein levels post-transcriptionally to facilitate transformation.

### REFERENCES

Anand, A., Krichevsky, A., Schornack, S., Lahaye, T., Tzfira, T., Tang, Y., Citovsky, V., and Mysore K. S. (2007). Arabidopsis VirE2 INTERACTING PROTEIN 2 is required for Agrobacterium T-DNA integration in plants. Plant Cell 19, 1695-1708.
Anders, S., Pyl, P. T., and Huber, W. (2015). HTSeq-a Python framework to work with high-throughput sequencing data. Bioinformatics 31, 166-169.
Ashburner, M., Ball, C. A., Blake, J. A., Botstein, D., Butler, H., Cherry, J. M., Davis, A. P., Dolinski, K., Dwight, S. S., Eppig, J. T., Harris, M. A., Hill, D. P., Issel-Tarver, L., Kasarkis, A., Lewis, S., Matese, J. C., Richardson, J. E., Ringwald, M., Rubin, G. M., and Sherlock, G. (2000). Gene ontology: Tool for the unification of biology. Nat. Genet. 25, 25-29.
Bakó L., Umeda, M., Tiburcio, A. F., Schell, J., and Koncz, C. (2003). The VirD2 pilot protein of Agrobacterium-transferred DNA interacts with the TATA box-binding protein and a nuclear protein kinase in plants. Proc. Natl. Acad. Sci. USA 100, 10108-10113.
Bhattacharjee, S., Lee, L.-Y., Oltmanns, H., Cao, H., Veena, Cuperus, J., and Gelvin, S. B. (2008). Atlmpa-4, an Arabidopsis importin a isoform, is preferentially involved in Agrobacterium-mediated plant transformation. Plant Cell 20, 2661-2680.
Chang, C. W., Counago, R. L., Williams, S. J., Boden, M., and Kobe, B. (2012). Crystal structure of rice importin-alpha and structural basis of its interaction with plant-specific nuclear localization signals. Plant Cell 24, 5074-5088.
Citovsky, V., De Vos, G., and Zambryski, P. (1988). Single-stranded DNA binding protein encoded by the virE locus of Agrobacterium tumefaciens. Science 240, 501-504.
Citovsky, V., Wong, M. L., and Zambryski, P. (1989). Cooperative interaction of Agrobacterium VirE2 protein with single-stranded DNA: implications for the T-DNA transfer process. Proc. Natl. Acad. Sci. USA 86, 1193-1197.
Citovsky, V., Zupan, J., Warnick, D., and Zambryski, P. (1992). Nuclear localization of Agrobacterium VirE2 protein in plant cells. Science 256, 1802-1805.
Citovsky, V., Warnick, D., and Zambryski, P. (1994). Nuclear import of Agrobacterium VirD2 and VirE2 proteins in maize and tobacco. Proc. Natl. Acad. Sci. USA 91, 3210-3214.
Citovsky, V., Kapelnikov, A., Oliel, S., Zakai, N., Rojas, M. R., Gilbertson, R. L., Tzfira, T., and Loyter, A. (2004). Protein interactions involved in nuclear import of the Agrobacterium VirE2 protein in vivo and in vitro. J. Biol. Chem. 279, 29528-29533.
Citovsky, V., Lee, L.-Y., Vyas, S., Glick, E., Chen, M.-H., Vainstein, A., Gafni, Y., Gelvin, S. B. and Tzfira, T. (2006). Subcellular localization of interacting proteins by bimolecular fluorescence complementation in planta. J. Mol. Biol. 362, 1120-1131.

Clough, S. J., and Bent, A. F. (1998). Floral dip: A simplified method for Agrobacterium-mediated transformation of Arabidopsis thaliana. Plant J. 16, 735-743.
Cox, J., and Mann, M. (2008). MaxQuant enables high peptide identification rates, individualized p.p.b.range mass accuracies and proteome-wide protein quantification. Nat. Biotechnol. 26, 1367-1372.
Cox, J., Neuhauser, N., Michalski, A., Scheltema, R. A., Olsen, J. V., and Mann, M. (2011). Andromeda: A peptide search engine integrated into the MaxQuant environment. J. Proteome Res. 10, 1794-1805.
Cox, J., Hein, M. Y., Luber, C. A., Paron, I., Nagaraj, N., and Mann, M. (2014). Accurate proteome-wide label-free quantification by delayed normalization and maximal peptide ratio extraction, termed MaxLFQ. Mol. Cell Proteomics 13, 2513-2526.
Crane, Y. M., and Gelvin, S. B. (2007). RNAi-mediated gene silencing reveals involvement of Arabidopsis chromatin-related genes in Agrobacterium-mediated root transformation. Proc. Natl. Acad. Sci. USA 104, 15156-15161.
Das, A. (1988). Agrobacterium tumefaciens virE operon encodes a single-stranded DNA-binding protein. Proc. Natl. Acad. Sci. USA 85, 2909-2913.
Deng, W., Chen, L., Wood, D. W., Metcalfe, T., Liang, X., Gordon, M. P., Comai, L, and Nester, E. W. (1998). Agrobacterium VirD2 protein interacts with plant host cyclophilins. Proc. Natl. Acad. Sci. USA 95, 7040-7045.
Ditt, R. F., Kerr, K. F., de Figueiredo, P. Delrow, J., Comai, L., and Nester, E. W. (2006). The Arabidopsis thaliana transcriptome in response to Agrobacterium tumefaciens. Mol. Plant-Microbe Interact. 19, 665-681.
Djamei, A., Pitzschke, A., Nakagami, H., Rajh, I., and Hirt, H. (2007). Trojan horse strategy in Agrobacterium transformation: Abusing MAPK defense signaling. Science 318, 453-456.
Dobin, A., Davis, C. A., Schlesinger, F., Drenkow, J., Zaleski, C., Jha, S., Batut, P., Chaisson, M., and Gingeras, T. R. (2013). STAR: Ultrafast universal RNA-seq aligner. Bioinformatics 29, 15-21.
Dombek, P., and Ream, W. (1997). Functional domains of Agrobacterium tumefaciens single-stranded DNA-binding protein VirE2. J. Bacteriol. 179, 1165-1173.
Du, Z., Zhou, X., Ling, Y., Zhang, Z., and Su, Z. (2010). agriGO: A GO analysis toolkit for the agricultural community. Nucl. Acids Res. 38. Web Server issue doi:10.1093/nar/gkq310.
Duan, K., Willig, C. J., De Tar, J. R., Spollen, W. G., and Zhang, Z. J. (2018). Transcriptomic analysis of Arabidopsis seedlings in response to Agrobacterium-mediated transformation process. Mol. Plant-Microbe Interact. 31, 445-459.
Durfee, T., Nelson, R., Baldwin, S., Plunkett, G., Burland, V., Mau, B., Petrosino, J. F., Qin, X., Muzny, D. M., Ayele, M., Gibbs, R. A., Csörgo, B., Pósfai, G., Weinstock, G. M., and Blattner, F. R. (2008). The complete genome sequence of Escherichia coli DH10B: Insights into the biology of the laboratory workhorse. J. Bacteriol. 190, 2597-2606.
Gaspar, Y. M., Nam, J., Schultz, C. J., Lee, L.-Y., Gilson, P. R., Gelvin, S. B., and Bacic, A. (2004). Characterization of the Arabidopsis lysine-rich arabinogalactan-protein AtAGP17 mutant (rat1) that results in a decreased efficiency of Agrobacterium transformation. Plant Physiol. 135, 2162-2171.
Gelvin, S. B. (2003). Agrobacterium-mediated plant transformation: The biology behind the “gene-jockeying” tool. Microbiol. Mol. Biol. Rev. 67, 16-37.
Gelvin, S. B. (2012). Traversing the cell: Agrobacterium T-DNA's journey to the host genome. Front. Plant Sci. 3, 1-11.
Gietl, C., Koukolikova-Nicola, Z., and Hohn, B. (1987). Mobilization of T-DNA from Agrobacterium to plant cells involves a protein that binds single-stranded DNA. Proc. Natl. Acad. Sci. USA 84, 9006-9010.
Grange, W., Duckely, M., Husale, S., Jacob, S., Engel, A., and Hegner, M. (2008), VirE2: A unique ssDNA-compacting molecular machine. PLoS Biol. 6, e44. doi:10.1371/journal.pbio.0060044
Hiei, Y., Ishida, Y., and Komari, T. (2014). Progress of cereal transformation technology mediated by Agrobacterium tumefaciens. Front. Plant. Sci. 5, 628.
Hood, E. E., Gelvin, S. B., Melchers, L. S., and Hoekema, A. (1993). New Agrobacterium helper plasmids for gene transfer to plants. Transgen. Res. 2, 208-218.
Howard, E., and Citovsky, V. (1990). The emerging structure of the Agrobacterium T-DNA transfer complex. BioEssays 12, 103-108.
Koncz, C., and Schell, J. (1986). The promoter of TL-DNA gene 5 controls the tissue-specific expression of chimaeric genes carried by a novel type of Agrobacterium binary vector. Mol. Gen. Genet. 204, 383-396.
Krueger, F. (2017). Babraham Bioinformatics. Trim Galore! 2017, 0153. doi: 10.1007/s10439 010-0153-9.Impact.
Lacroix, B., and Citovsky, V. (2013). The roles of bacterial and host plant factors in Agrobacterium-mediated genetic transformation. Int. J. Dev. Biol. 57, 467-481.
Lacroix, B., and Citovsky, V. (2019). Pathways of DNA transfer to plants from Agrobacterium tumefaciens and related bacterial species. Annu. Rev. Phytopathol. 57, 231-251.
Lapham, R., Lee L.-Y., Tsugama D., Lee S., Mengiste T., and Gelvin S. B. (2018). VIP1 and its homologs are not required for Agrobacterium-mediated transformation, but play a role in Botrytis and salt stress responses. Frontiers Plant Sci. 9, 1-15.
Lee, L.-Y., Fang, M.-J., Kuang, L.-Y., and Gelvin, S. B. (2008). Vectors for multi-color bimolecular fluorescence complementation to investigate protein-protein interactions in living plant cells. Plant Methods 4, 24.
Lee, L.-Y., Wu, F.-H., Hsu, C.-T., Shen, S.-C., Yeh, H.-Y., Liao, D.-C., Fang, M.-J., Liu, N.-T., Yen, Y.-C., Dokládal, L., Sýkorovà, E., Gelvin, S. B., and Lin, C.-S. (2012). Screening of a cDNA library for protein-protein interactions directly in planta. Plant Cell 24, 1746-1759.
Li, J., Krichevsky, A., Vaidya, M., Tzfira, T., and Citovsky, V. (2005). Uncoupling of the functions of the Arabidopsis VIP1 protein in transient and stable plant genetic transformation by Agrobacterium. Proc. Natl. Acad. Sci. USA 102, 5733-5738.
Li, X., and Pan, S. Q. (2017). Agrobacterium delivers VirE2 protein into host cells via clatherin-mediated endocytosis. Sci. Adv. 3, e1601528.
Li, X., Yang, Q., Tu, H., Lim, Z., and Pan, S. Q. (2014). Direct visualization of Agrobacterium-delivered VirE2 in recipient cells. Plant J. 77, 487-495.
Li, X., Yang, Q., Peng, L., Tu, H., Lee, L.-Y., Gelvin, S. B., and Pan, S. Q. (2020). Agrobacterium-delivered VirE2 interacts with host nucleoporin CG1 to facilitate the nuclear import of VirE2-coated T-complex. Proc. Natl. Acad. Sci. USA 117, 26389-26397.
Love, M. I., Huber, W., and Anders, S. (2014). Moderated estimation of fold change and dispersion for RNA-seq data with DESeq2. Genome Biol. 15:550
Mi, H., Muruganujan, A., Casagrande, J. T., and Thomas, P. D. (2013). Large-scale gene function analysis with the PANTHER classification system. Nat. Protoc. 8, 1551-1566.
Nam, J., Mysore, K. S., Zheng, C., Knue, M. K., Matthyssee, A. G., and Gelvin, S. B. (1999). Identification of T-DNA tagged Arabidopsis mutants that are resistant to transformation by Agrobacterium. Mol. Gen. Genet. 261, 429-438.
Narasimhulu, S. B., Deng, X.-B., Sarria, R., and Gelvin, S. B. (1996). Early transcription of Agrobacterium T-DNA genes in tobacco and maize. Plant Cell 8, 873-886.
Nester, E. W. (2015). Agrobacterium: Nature's genetic engineer. Front. Plant Sci. 5, 730.
Park, S.-Y., Yin, X., Duan, K., Gelvin, S. B., and Zhang, Z. J. (2014). Heat shock protein 90.1 plays a role in Agrobacterium-mediated plant transformation. Mol. Plant 7, 1793-1796.
Pitzschke, A., Djamei, A., Teige, M., Hirt, H. (2009). VIP1 response elements mediate mitogen-activated protein kinase 3-induced stress gene expression. Proc. Natl. Acad. Sci. USA 106, 18414-18419.
Pitzschke, A., and Hirt, H. (2010). New insights into an old story: Agrobacterium-induced tumour formation in plants by plant transformation. EMBO J. 29, 1021-1032.
Robinson, M. D., McCarthy, D. J., and Smyth, G. K. (2010). edgeR: A Bioconductor package for differential expression analysis of digital gene expression data. Bioinformatics 26, 139-140.
Rossi, L., Hohn, B., and Tinland, B. (1996). Integration of complete transferred DNA units is dependent on the activity of virulence E2 protein of Agrobacterium tumefaciens. Proc. Natl. Acad. Sci. USA 93, 126-130.
Roushan, M. R., de Zeeuw, M. A. M., Hooykaas, P. J. J., and van Heusden, G. P. H. (2018). Application of phiLOV2.1 as a fluorescent marker for visualization of Agrobacterium effector protein translocation. Plant J. 96, 685-699.
Schultz, C. J., Rumsewicz, M. P., Johnson, K. L., Jones, B. J., Gaspar, Y. M., and Bacic, A. (2002). Using genomic resources to guide research directions. The arabinogalactan-protein gene family as a test case. Plant Physiol. 129, 1448-1463.
Sciaky, D., Montoya, A. L., and Chilton, M.-D. (1978). Fingerprints of Agrobacterium Ti plasmids. Plasmid 1, 238-253.
Sen, P., Pazour, G. J., Anderson, D., and Das, A. (1989). Cooperative binding of Agrobacterium tumefaciens VirE2 protein to single-stranded DNA. J. Bacteriol. 171, 2573-2580.
Shi, Y., Lee L.-Y., and Gelvin S. B. (2014). Is VIP1 important for Agrobacterium-mediated transformation? Plant J. 79, 848-860.
Simone, M., McCullen, C. A., Stahl, L. E., and Binns, A. N. (2001). The carboxy-terminus of VirE2 from Agrobacterium tumefaciens is required for its transport to host cells by the virB-encoded type IV transport system. Mol. Microbiol. 41, 1283-1293.
Stachel, S. E., and Nester, E. W. (1986). The genetic and transcriptional organization of the vir region of the A6 Ti plasmid of Agrobacterium tumefaciens. EMBO J. 5, 1445-1454.
Tao, Y., Rao, P. K., Bhattacharjee, S., and Gelvin, S. B. (2004). Expression of plant protein phosphatase 2C interferes with nuclear import of the Agrobacterium T-complex protein VirD2. Proc. Natl. Acad. Sci. USA 101, 5164-5169.
Tenea, G. N., Spantzel, J., Lee L.-Y., Zhu, Y., Lin, K., Johnson, S. J., and Gelvin, S. B. (2009). Over-expression of several Arabidopsis histone genes increases Agrobacterium-mediated transformation and transgene expression in plants. Plant Cell 21, 3350-3367.
Tinland, B., Hohn, B., and Puchta, H. (1994). Agrobacterium tumefaciens transfers single-stranded transferred DNA (T-DNA) into the plant cell nucleus. Proc. Natl. Acad. Sci. USA 91, 8000-8004.
Trapnell, C., Williams, B. A., Pertea, G., Mortazavi, A., Kwan, G., van Baren, M. J., Salzberg, S. L., Wold, B. J., and Pachter, L. (2010). Transcript assembly and quantification by RNA-Seq reveals unannotated transcripts and isoform switching during cell differentiation. Nat. Biotechnol. 28, 511-515.

Trapnell C., Roberts A., Goff, L., Pertea, G., Kim, D., Kelley, D. R., Pimentel, H., Salzburg, S. L., Rinn, J. L., and Pachter, L. (2012). Differential gene and transcript expression analysis of RNA-seq experiments with TopHat and Cufflinks. Nat. Protoc. 7, 562-578.
Tsugama, D., Liu, S., and Takano, T. (2012). A bZIP protein, VIP1, is a regulator of osmosensory signaling in Arabidopsis. Plant Physiol. 159, 144-155.
Tsugama, D., Liu, S., and Takano, T. (2014). Analysis of functions of VIP1 and its close homologs in osmosensory responses of Arabidopsis thaliana. PLoS One 9(8), e103930.
Tsugama, D., Liu, S., and Takano, T. (2016). The bZIP protein VIP1 is involved in touch responses in Arabidopsis roots. Plant Physiol. 171, 1355-1365.
Tzfira, T., Vaidya, M., and Citovsky, V. (2001). VI P1, an Arabidopsis protein that interacts with Agrobacterium VirE2, is involved in VirE2 nuclear import and Agrobacterium infectivity. EMBO J. 20, 3596-3607.
Tzfira, T., and Citovsky, V. (2001). Comparison between nuclear localization of nopaline- and octopine-specific Agrobacterium VirE2 proteins in plant, yeast and mammalian cells. Mol. Plant Pathol. 2, 171-176.
Tzfira, T., Tian, G.-W., Lacroiz, B., Vyas, S., Li, J., Leitner-Dagab, Y., Krishevsky, A., Taylor, T., Vainstein, A., and Citovsky, V. (2005). pSAT vectors: A modular series of plasmids for autofluorescent protein tagging and expression of multiple genes in plants. Plant Mol. Biol. 57, 503-516.
Van Eck, J. (2018). Genome editing and plant transformation of solanaceous food crops. Curr. Opin. Biotechnol. 49, 35-41.
Van Larebeke, N., Engler, G., Holsters, M., Van Den Elsacker, S., Zaenen, I., Schilperoort, R. A., and Schell, J. (1974). Large plasmid in Agrobacterium tumefaciens essential for crown gall-inducing ability. Nature 252, 169-170.
Veena, Jiang, H., Doerge, R. W., and Gelvin, S. B. (2003). Transfer of T-DNA and Vir proteins to plant cells by Agrobacterium tumefaciens induces expression of host genes involved in mediating transformation and suppresses host defense gene expression. Plant J. 35, 219-236.
Wu, L., Chen, H., Curtis, C., and Fu, Z. Q. (2014). Go in for the kill: How plants deploy effector-triggered immunity to combat pathogens. Virulence 5, 710-721.
Yusibov, V. M., Steck, T. R., Gupta, V., and Gelvin, S. B. (1994). Association of single-stranded transferred DNA from Agrobacterium tumefaciens with tobacco cells. Proc. Natl. Acad. Sci. USA 91, 2994-2998.
Zheng, Z., Qamar, S. A., Chen, Z., and Mengiste, T. (2006). Arabidopsis WRKY33 transcription factor is required for resistance to necrotrophic fungal pathogens. Plant J. 48, 592-605.
Ziemienowicz, A., Merkle, T., Schoumacher, F., Hohn, B., and Rossi, L. (2001). Import of Agrobacterium T-DNA into plant nuclei: two distinct functions of VirD2 and VirE2 proteins. Plant Cell 13, 369-383.
Zuo, J., Niu, Q.-W., and Chua, N.-H. (2000). An estrogen receptor-based system transactivator XVE mediates highly inducible gene expression in transgenic plants. Plant J. 24, 265-273.

What is claimed is:

1. A transgenic plant or plant cell comprising an overexpressed plant gene or protein that is upregulated by VirE2 gene or VirE2 protein in the cytoplasm to facilitate transformation.

2. The transgenic plant or plant cell of claim 1, wherein said upregulated plant gene or protein is selected from the group consisting of a transcription factor, an arabinogalactan protein (AGP) gene or protein, a heat shock protein (HSP) transcript or protein, a histone or histone modifying enzyme or its variants, and a cyclophilin protein.

3. The transgenic plant or plant cell of claim 2, wherein said transcription factor is WRKY33.

4. The transgenic plant or plant cell of claim 2, wherein said arabinogalactan protein (AGP) gene or protein is AGP17 or AGP31.

5. The transgenic plant or plant cell of claim 2, wherein said HSP transcript or protein is HSP90.

6. The transgenic plant or plant cell of claim 2, wherein said histone or histone modifying enzyme is histone H2A2 (HTA2) or its variants, histone H4 (HIS4 or HFO4), histone deacetylase HD2C (HDT3) or HDA3 (HDT1).

7. The transgenic plant or plant cell of claim 2, wherein said cyclophilin protein is ROC2 or ROC3.

8. A transgenic plant or plant cell comprising a downexpressed or knockout plant gene or protein that is downregulated by VirE2 gene or VirE2 protein in the cytoplasm to facilitate transformation.

9. The transgenic plant or plant cell of claim 8, wherein said downregulated plant gene or protein is alcohol dehydrogenase (ADH1) or a protein phosphatase 2C 25 (PP2C25).

10. A transgenic plant or plant cell comprising overexpressed VirE2 gene or VirE2 protein in the plant cytoplasm, wherein said VirE2 gene or VirE2 protein alters expression of a plant gene or protein to facilitate transformation in response to VirE2 gene or VirE2 protein induction.

11. The transgenic plant or plant cell of claim 10, wherein the expression of said plant gene or protein is upregulated in response to VirE2 gene or VirE2 protein induction.

12. The transgenic plant or plant cell of claim 11, wherein said plant gene or protein is selected from the group consisting of a transcription factor, an arabinogalactan protein (AGP) gene or protein, a heat shock protein (HSP) transcript or protein, a histone or histone modifying enzyme, and a cyclophilin protein.

13. The transgenic plant or plant cell of claim 12, wherein said transcription factor is WRKY33.

14. The transgenic plant or plant cell of claim 12, wherein said arabinogalactan protein (AGP) gene or protein is AGP17 or AGP31.

15. The transgenic plant or plant cell of claim 12, wherein said HSP transcript or protein is HSP90.

16. The transgenic plant or plant cell of claim 12, wherein said histone or histone modifying enzyme is histone H2A2 (HTA2) or its variants, histone H4 (HIS4 or HFO4), histone deacetylase HD2C (HDT3) or HDA3 (HDT1).

17. The transgenic plant or plant cell of claim 12, wherein said cyclophilin protein is ROC2 or ROC3.

18. The transgenic plant or plant cell of claim 10, wherein the expression of said plant gene or protein is downregulated in response to VirE2 gene or VirE2 protein induction.

19. The transgenic plant or plant cell of claim 13, wherein said plant gene or protein is alcohol dehydrogenase (ADH1) or a protein phosphatase 2C 25 (PP2C25).


SEQUENCE LISTING (84 sequences)

SEQ ID NO: 1 - DNA, 26 bp, Artificial Sequence
  Description of Artificial Sequence Synthetic primer
cttggtgaagcagctgacaaatactc

SEQ ID NO: 2 - DNA, 26 bp, Artificial Sequence
  Description of Artificial Sequence Synthetic primer
agactggtgatttttgcggactctag

SEQ ID NO: 3 - DNA, 25 bp, Artificial Sequence
  Description of Artificial Sequence Synthetic primer
cggggttgtggaaaagtacatgaac

SEQ ID NO: 4 - DNA, 22 bp, Artificial Sequence
  Description of Artificial Sequence Synthetic primer
gcttcaagcacccatggtgatg

SEQ ID NO: 5 - DNA, 23 bp, Artificial Sequence
  Description of Artificial Sequence Synthetic primer
tgacccgaacttcgacctttacc

SEQ ID NO: 6 - DNA, 28 bp, Artificial Sequence
  Description of Artificial Sequence Synthetic primer
tcaatgaaccgctttgagtagcgtatac

SEQ ID NO: 7 - DNA, 26 bp, Artificial Sequence
  Description of Artificial Sequence Synthetic primer
aagtcactcaaacactgacgtgaacc

SEQ ID NO: 8 - DNA, 25 bp, Artificial Sequence
  Description of Artificial Sequence Synthetic primer
cgtccttcaccacggcaatttcatg

SEQ ID NO: 9 - DNA, 29 bp, Artificial Sequence
  Description of Artificial Sequence Synthetic primer
cactatactcaggttgtgtggagaaactc

SEQ ID NO: 10 - DNA, 20 bp, Artificial Sequence
  Description of Artificial Sequence Synthetic primer
ccactcgccaacccagttac

SEQ ID NO: 11 - DNA, 25 bp, Artificial Sequence
  Description of Artificial Sequence Synthetic primer
gagctggaggtcgagtctttagaac

SEQ ID NO: 12 - DNA, 29 bp, Artificial Sequence
  Description of Artificial Sequence Synthetic primer
cttattctacgaggaagagacgacagaag

SEQ ID NO: 13 - DNA, 28 bp, Artificial Sequence
  Description of Artificial Sequence Synthetic primer
tccttcatcagctagaagaccgaacatg

SEQ ID NO: 14 - DNA, 21 bp, Artificial Sequence
  Description of Artificial Sequence Synthetic primer
ccgagccaatggggtcacttc

SEQ ID NO: 15 - DNA, 26 bp, Artificial Sequence
  Description of Artificial Sequence Synthetic primer
aaagatctatggaggcaatgaagatg

SEQ ID NO: 16 - DNA, 29 bp, Artificial Sequence
  Description of Artificial Sequence Synthetic primer
ttcttaagtcaaaagatgaaaccagatgc

SEQ ID NO: 17 - DNA, 27 bp, Artificial Sequence
  Description of Artificial Sequence Synthetic primer
gaagagcattactatggagcggaatgg

SEQ ID NO: 18 - DNA, 25 bp, Artificial Sequence
  Description of Artificial Sequence Synthetic primer
cttcactagacatgagccggagatc

SEQ ID NO: 19 - DNA, 30 bp, Artificial Sequence
  Description of Artificial Sequence Synthetic primer
ctgcattttggagaaagacctaatctcaag

SEQ ID NO: 20 - DNA, 30 bp, Artificial Sequence
  Description of Artificial Sequence Synthetic primer
agagttatatacgcaatcaccagctgaaac

SEQ ID NO: 21 - DNA, 21 bp, Artificial Sequence
  Description of Artificial Sequence Synthetic primer
gagtcgtccgcttggtctaac

SEQ ID NO: 22 - DNA, 26 bp, Artificial Sequence
  Description of Artificial Sequence Synthetic primer
cttggacctgagtgcttaacaaatcg

SEQ ID NO: 23 - DNA, 27 bp, Artificial Sequence
  Description of Artificial Sequence Synthetic primer
gctaggattcacaggatgttgaagttg

SEQ ID NO: 24 - DNA, 25 bp, Artificial Sequence
  Description of Artificial Sequence Synthetic primer
acttcctccatcttgctctcttcag

SEQ ID NO: 25 - DNA, 22 bp, Artificial Sequence
  Description of Artificial Sequence Synthetic primer
gtcggacaaccgctcataacac

SEQ ID NO: 26 - DNA, 27 bp, Artificial Sequence
  Description of Artificial Sequence Synthetic primer
agaacaagtgaacaacaccgtttatcc

SEQ ID NO: 27 - DNA, 26 bp, Artificial Sequence
  Description of Artificial Sequence Synthetic primer
acaagtcaacctttctcctcgtgtag

SEQ ID NO: 28 - DNA, 24 bp, Artificial Sequence
  Description of Artificial Sequence Synthetic primer
gcttggaagacccatgcaagatag

SEQ ID NO: 29 - DNA, 22 bp, Artificial Sequence
  Description of Artificial Sequence Synthetic primer
tggttcacgtagtgggccatcg

SEQ ID NO: 30 - DNA, 31 bp, Artificial Sequence
  Description of Artificial Sequence Synthetic primer
aagagctcctagctatatattctggagactc

SEQ ID NO: 31 - DNA, 34 bp, Artificial Sequence
  Description of Artificial Sequence Synthetic primer
ttccgcgggattaactgttaaaagattcaaaaac

SEQ ID NO: 32 - DNA, 21 bp, Artificial Sequence
  Description of Artificial Sequence Synthetic primer
atgtgttacgcagtttcgtcc

SEQ ID NO: 33 - DNA, 21 bp, Artificial Sequence
  Description of Artificial Sequence Synthetic primer
agctttgcttcatgttcttgg

SEQ ID NO: 34 - DNA, 27 bp, Artificial Sequence
  Description of Artificial Sequence Synthetic primer
aaagatctatggtggcttttgcaacag

SEQ ID NO: 35 - DNA, 29 bp, Artificial Sequence
  Description of Artificial Sequence Synthetic primer
ttcttaagttaagtctgtgcacggactag

SEQ ID NO: 36 - DNA, 29 bp, Artificial Sequence
  Description of Artificial Sequence Synthetic primer
aaagatctatgtctcaatcaatctcctcc

SEQ ID NO: 37 - DNA, 29 bp, Artificial Sequence
  Description of Artificial Sequence Synthetic primer
ttcttaagttaattagcagatggctcctc

SEQ ID NO: 38 - DNA, 21 bp, Artificial Sequence
  Description of Artificial Sequence Synthetic primer
catttcattaatggctcgctc

SEQ ID NO: 39 - DNA, 21 bp, Artificial Sequence
  Description of Artificial Sequence Synthetic primer
attgctgttatggccacagac

SEQ ID NO: 40 - DNA, 21 bp, Artificial Sequence
  Description of Artificial Sequence Synthetic primer
tttaggagttgtgcccatgtc

SEQ ID NO: 41 - DNA, 25 bp, Artificial Sequence
  Description of Artificial Sequence Synthetic primer
ccttaacgtgtcataaatcaattcc

SEQ ID NO: 42 - DNA, 21 bp, Artificial Sequence
  Description of Artificial Sequence Synthetic primer
cgaggttaaaattccgaaagg

SEQ ID NO: 43 - DNA, 21 bp, Artificial Sequence
  Description of Artificial Sequence Synthetic primer
gtccgcaatacgtaaaactcg

SEQ ID NO: 44 - DNA, 23 bp, Artificial Sequence
  Description of Artificial Sequence Synthetic primer
acccggtggataaaatcgagttc

SEQ ID NO: 45 - DNA, 23 bp, Artificial Sequence
  Description of Artificial Sequence Synthetic primer
tcaagcgtccagacttcagattg

SEQ ID NO: 46 - DNA, 21 bp, Artificial Sequence
  Description of Artificial Sequence Synthetic primer
tgttgcatttgtggacaagac

SEQ ID NO: 47 - DNA, 21 bp, Artificial Sequence
  Description of Artificial Sequence Synthetic primer
tggagtgatctcgtaacggac

SEQ ID NO: 48 - DNA, 23 bp, Artificial Sequence
  Description of Artificial Sequence Synthetic primer
tgagaagagagcaaagaaacttc

SEQ ID NO: 49 - DNA, 23 bp, Artificial Sequence
  Description of Artificial Sequence Synthetic primer
catgtggaaacaaacgtatgaag

SEQ ID NO: 50 - DNA, 28 bp, Artificial Sequence
  Description of Artificial Sequence Synthetic primer
ataataacgctgcggacatctacatttt

SEQ ID NO: 51 - DNA, 25 bp, Artificial Sequence
  Description of Artificial Sequence Synthetic primer
tctattacattcgcggcaatattcg

SEQ ID NO: 52 - DNA, 25 bp, Artificial Sequence
  Description of Artificial Sequence Synthetic primer
gctatacgtgtagggctcataagac

SEQ ID NO: 53 - DNA, 29 bp, Artificial Sequence
  Description of Artificial Sequence Synthetic primer
atgaagaatctttgttgggtttttctgtc

SEQ ID NO: 54 - DNA, 27 bp, Artificial Sequence
  Description of Artificial Sequence Synthetic primer
gaacgatcataaacatctttcgggtac

SEQ ID NO: 55 - DNA, 34 bp, Artificial Sequence
  Description of Artificial Sequence Synthetic primer
aaagatctatggcttcctctatgctctcctccgc

SEQ ID NO: 56 - DNA, 37 bp, Artificial Sequence
  Description of Artificial Sequence Synthetic primer
ttggtaccaagaaattaagcttcggtgaagcttgggg

SEQ ID NO: 57 - DNA, 26 bp, Artificial Sequence
  Description of Artificial Sequence Synthetic primer
aagagctcatggatttctccggtttg

SEQ ID NO: 58 - DNA, 28 bp, Artificial Sequence
  Description of Artificial Sequence Synthetic primer
ttggtaccctatggttttcgttccaagg

SEQ ID NO: 59 - DNA, 21 bp, Artificial Sequence
  Description of Artificial Sequence Synthetic primer
caccaaagagagctccacaag

SEQ ID NO: 60 - DNA, 21 bp, Artificial Sequence
  Description of Artificial Sequence Synthetic primer
ggctctattggaacctccttg

SEQ ID NO: 61 - DNA, 21 bp, Artificial Sequence
  Description of Artificial Sequence Synthetic primer
catcgtcaccacaatctttcc

SEQ ID NO: 62 - DNA, 21 bp, Artificial Sequence
  Description of Artificial Sequence Synthetic primer
ggacaaaagtttgcatatggc

SEQ ID NO: 63 - DNA, 29 bp, Artificial Sequence
  Description of Artificial Sequence Synthetic primer
gttggttgcacatcatcatgggaatcttg

SEQ ID NO: 64 - DNA, 20 bp, Artificial Sequence
  Description of Artificial Sequence Synthetic primer
ggcgtccaccctctgacttg

SEQ ID NO: 65 - DNA, 27 bp, Artificial Sequence
  Description of Artificial Sequence Synthetic primer
aaagatctatggtggagaagtcaggag

SEQ ID NO: 66 - DNA, 30 bp, Artificial Sequence
  Description of Artificial Sequence Synthetic primer
tccttaagattctcactcaccatattcagg

SEQ ID NO: 67 - DNA, 21 bp, Artificial Sequence
  Description of Artificial Sequence Synthetic primer
tgaacgtctgtgaagttcacg

SEQ ID NO: 68 - DNA, 21 bp, Artificial Sequence
  Description of Artificial Sequence Synthetic primer
tgccggttttgtattcttgtc

SEQ ID NO: 69 - DNA, 21 bp, Artificial Sequence
  Description of Artificial Sequence Synthetic primer
caagagctgaaagcctcaaac

SEQ ID NO: 70 - DNA, 21 bp, Artificial Sequence
  Description of Artificial Sequence Synthetic primer
ttaccaggatgagatgatcgg

SEQ ID NO: 71 - DNA, 21 bp, Artificial Sequence
  Description of Artificial Sequence Synthetic primer
caccaatcttcatggagatcg

SEQ ID NO: 72 - DNA, 21 bp, Artificial Sequence
  Description of Artificial Sequence Synthetic primer
gattaatttcggccaatgctc

SEQ ID NO: 73 - DNA, 21 bp, Artificial Sequence
  Description of Artificial Sequence Synthetic primer
cgatgggtacaccgattactg

SEQ ID NO: 74 - DNA, 21 bp, Artificial Sequence
  Description of Artificial Sequence Synthetic primer
aaagatcggcaacacatgatc

SEQ ID NO: 75 - DNA, 28 bp, Artificial Sequence
  Description of Artificial Sequence Synthetic primer
aagaattcatgcatttctcttcgtcttc

SEQ ID NO: 76 - DNA, 29 bp, Artificial Sequence
  Description of Artificial Sequence Synthetic primer
aaggatcctcacatagagctaacaaagtc

SEQ ID NO: 77 - DNA, 28 bp, Artificial Sequence
  Description of Artificial Sequence Synthetic primer
aaagatctatgggtttcattggtaagag

SEQ ID NO: 78 - DNA, 24 bp, Artificial Sequence
  Description of Artificial Sequence Synthetic primer
aaggatcctcatttggggcaagac

SEQ ID NO: 79 - DNA, 27 bp, Artificial Sequence
  Description of Artificial Sequence Synthetic primer
aagaattcatggagttctggggaattg

SEQ ID NO: 80 - DNA, 23 bp, Artificial Sequence
  Description of Artificial Sequence Synthetic primer
aaggatcctcacttggcagcagc

SEQ ID NO: 81 - DNA, 24 bp, Artificial Sequence
  Description of Artificial Sequence Synthetic primer
aaagatctatggagttctggggtg

SEQ ID NO: 82 - DNA, 24 bp, Artificial Sequence
  Description of Artificial Sequence Synthetic primer
aaggatcctcaagcagctgcactg

SEQ ID NO: 83 - DNA, 26 bp, Artificial Sequence
  Description of Artificial Sequence Synthetic primer
aagaattcatggcgaatcctaaagtc

SEQ ID NO: 84 - DNA, 29 bp, Artificial Sequence
  Description of Artificial Sequence Synthetic primer
aaggatccttatgaacttgggttcttgag